(19) United States
(12) Patent Application Publication
(10) Pub. No.: US 2013/0039887 A1
(43) Pub. Date: Feb. 14, 2013

(54) Methods of Identifying and Using Anti-Viral Compounds

(71) Applicants: Shawn P. Iadonato, Seattle, WA (US); Kristin Bedard, Seattle, WA (US); Michael Gale, Seattle, WA (US)

(72) Inventors: Shawn P. Iadonato, Seattle, WA (US); Kristin Bedard, Seattle, WA (US); Michael Gale, Seattle, WA (US)

(73) Assignee: KINETA, INC., Seattle, WA (US)

(21) Appl. No.: 13/642,801

(22) PCT Filed: Apr. 20, 2011
(86) PCT No.: PCT/US11/33257

§ 371 Date: Oct. 22, 2012

Related U.S. Application Data
(60) Provisional application No. 61/327,367, filed on Apr. 23, 2010.

(51) Int. Cl. C12Q 1/70; A61K 38/21; G01N 21/76; A61P 31/18; A61P 31/22; A61P 31/16; C40B 30/10; A61P 31/12

(52) U.S. Cl. 424/85.7; 435/5; 506/12

(57) ABSTRACT

Disclosed herein are methods for identifying compounds for the treatment of viral infection, including RNA viral infection and uses of the compounds as pharmaceutical compositions. The identified compounds modulate the RIG-I pathway in vertebrate cells.

### FIELD OF THE DISCLOSURE

Methods disclosed herein are useful for identifying compounds for treating viral infection in vertebrates, including RNA viral infections. The identified compounds can modulate the RIG-I pathway.

### BACKGROUND OF THE DISCLOSURE

As a group, RNA viruses represent an enormous public health problem in the U.S. and worldwide. Well-known RNA viruses include influenza virus (including the avian and swine isolates), hepatitis C virus (HCV), West Nile virus, SARS-coronavirus, respiratory syncytial virus (RSV), and human immunodeficiency virus (HIV).

More than 170 million people worldwide are infected by HCV, and 130 million of those are chronic carriers at risk of developing chronic liver diseases (cirrhosis, carcinoma, and liver failure). As such, HCV is responsible for two thirds of all liver transplants in the developed world. Recent studies show that the death rate from HCV infection is rising due to the increasing age of chronically infected patients. Likewise seasonal flu infects 5-20% of the population resulting in 200,000 hospitalizations and 36,000 deaths each year.

Compared to influenza and HCV, West Nile virus causes the lowest number of infections, 981 in the United States in 2010. Twenty percent of infected patients develop a severe form of the disease, resulting in a 4.5% mortality rate. Unlike influenza and HCV, there are no approved therapies for the treatment of West Nile virus infection, and it is a high-priority pathogen for drug development due to its potential as a bioterrorist agent.

Among the RNA viruses listed, vaccines exist only for influenza virus. Accordingly, drug therapy is essential to mitigate the significant morbidity and mortality associated with these viruses. Unfortunately, the number of antiviral drugs is limited, many are poorly effective, and nearly all are plagued by the rapid evolution of viral resistance and a limited spectrum of action. Moreover, treatments for acute influenza and HCV infections are only moderately effective. The standard of care for HCV infection, PEGylated interferon and ribavirin, is effective in only 50% of patients, and there are a number of dose-limiting side effects associated with the combined therapy. Both classes of acute influenza antivirals, adamantanes and neuraminidase inhibitors, are only effective within the first 48 hours after infection, thereby limiting the window of opportunity for treatment. High resistance to adamantanes already restricts their use, and massive stockpiling of neuraminidase inhibitors will eventually lead to overuse and the emergence of resistant strains of influenza.

Most drug development efforts against these viruses target viral proteins. This is a large part of the reason that current drugs are narrow in spectrum and subject to the emergence of viral resistance. Most RNA viruses have small genomes and many encode less than a dozen proteins. Viral targets are therefore limited. Based on the foregoing, there is an immense and unmet need for effective treatments against viral infections.

### SUMMARY OF THE DISCLOSURE

The present disclosure helps to meet the need for effective virus treatment methods by providing methods to identify structural classes of compounds that stimulate innate immune signaling. The identified structural classes of compounds shift the focus of viral drug development away from the targeting of viral proteins to the development of drugs that target and enhance the host's innate antiviral response. Such compounds and methods are likely to be more effective, less susceptible to the emergence of viral resistance, cause fewer side effects and be effective against a range of different viruses(1).

The RIG-I pathway is intimately involved in regulating the innate immune response to RNA virus infections. RIG-I agonists are expected to be useful for the treatment and/or prevention of infection by many viruses including, without limitation, HCV, influenza, and West Nile virus. Accordingly, the present disclosure relates to methods to identify compounds for treating and/or preventing viral infection, including infection by RNA viruses, wherein the compounds modulate the RIG-I pathway.

One embodiment includes a method of identifying a compound that modulates innate immunity, comprising the steps of: contacting at least one cell comprising a reporter gene under the control of a gene promoter responsive to innate immune activation with at least one putative innate immune response modulating compounds; and measuring reporter gene activation.

In another embodiment, the method further comprises selecting a compound that activates reporter gene expression above a selected threshold for further characterization. In another embodiment, the selected threshold is four standard deviations above a control level.

In another embodiment, the further characterization includes measuring nuclear translocation of transcription factors responsive to innate immune activation. In another embodiment, the measuring of nuclear translocation is by immunochemical assay.

In another embodiment, prior to contacting the compound is structurally selected for predicted binding to the ligand-binding domain of RIG-I.

In another embodiment, the cells are eukaryotic cells. In another embodiment, the eukaryotic cells are Huh7 cells.

In another embodiment, the reporter gene is luciferase.

Another embodiment includes a method comprising providing at least one eukaryotic cell comprising a reporter gene under the control of a gene promoter responsive to innate immune activation for identifying compounds that modulate innate immune responses.

In another embodiment, the cells are eukaryotic cells. In another embodiment, the eukaryotic cells are Huh7 cells.

In another embodiment, the reporter gene is luciferase.

Another embodiment includes a method of preventing or treating a viral infection in a vertebrate by administering to the vertebrate a compound identified by contacting at least one cell comprising a reporter gene under the control of a gene promoter responsive to innate immune activation with at least one putative innate immune response modulating compounds; wherein said viral infection is treated, reduced or prevented.

In another embodiment, the compound activates reporter gene expression above a selected threshold for further characterization. In another embodiment, the selected threshold is four standard deviations above a control level.

In another embodiment, the compound induces nuclear translocation of transcription factors responsive to innate immune activation.

In another embodiment, the viral infection is by a virus within one of the following families: Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae.

In another embodiment, the viral infection is influenza virus, Hepatitis C virus, West Nile virus, SARS-coronavirus, poliovirus, measles virus, Dengue virus, yellow fever virus, tick-borne encephalitis virus, Japanese encephalitis virus, St. Louis encephalitis virus, Murray Valley virus, Powassan virus, Rocio virus, Iouping-ill virus, Banzi virus, Ilheus virus, Kokobera virus, Kunjin virus, Alfuy virus, bovine diarrhea virus, Kyasanur forest disease virus or human immunodeficiency virus (HIV).

### BRIEF DESCRIPTION OF THE FIGURES

FIG. 1 shows transient expression and induction of ISG54 and ISG56 reporter constructs with Sendai virus and IFN.

FIG. 2 shows normalized luciferase expression under increasing concentrations of IFN.

FIG. 3 shows that stable luciferase cell lines show various induction with Sendai virus.

FIG. 4 shows the targeted library scatter plot after initial screen. Negative controls (no treatment-gray) and positive controls (Sendai infection) were included on each plate. The luciferase values for all compounds screened are shown in red. The line represents the threshold for identifying initial hits.

FIG. 5 shows that the majority of targeted set hits do not cause activation of the actin promoter.

FIG. 6 shows dose dependent activity of compounds from the targeted set in the ISG54 reporter assay.

FIG. 7 shows compound cytotoxicity in Huh7 cells using an MTS assay.

FIG. 8 shows IRF-3 translocation in Huh7 cells treated with compound. Cells are pre-treated with (10 or 20 μM) of compound for 24 hours and then stained for IRF-3. Mock treated cells show the majority of IRF-3 in the cytoplasm, Sendai infected cells have accumulated IRF-3 in the nucleus and compounds showed IRF-3 in the nucleus as well.

FIG. 9 shows HCV antiviral activity in the IF assay. Huh7 cells are pre-treated with compound for 24 hours, infected with HCV at a low MOI for 48 hours and then stained for HCV proteins. Mock infected cells show no background staining, and interferon completely blocks infection and serves as a positive control. The number of infected cells (stained green for HCV proteins) are counted on an inverted microscope. The number of HCV infected cells after treatment for each compound is shown in chart.

FIG. 10 shows the results of experiments in which Huh7 cells were pre-treated with compound at increasing concentrations 0-10 uM for 24 hours. Cells were then infected and analyzed for HCV foci as described.

FIG. 11 shows the results of experiments in which Huh7 cells were treated with 10 μM of compound for 24 hours and subsequently HCV infections were done as described in Example 2.

FIG. 12 is a histogram of luminescence data from a primary screen for ISG induction. A 20K diversity library was screened at 10 μM to identify compounds that induce ISG54 luciferase reporter activity (grey histogram, 1° Y axis). Negative (cells alone) and positive controls (Sendai virus infected cells) are represented as cumulative frequency histograms (2° Y axis). Yellow line indicates the 4 SD threshold used to identify positive hits (inset). RLU, Renilla-luciferase.

FIG. 13 shows characterization of compound KIN300, isolated from the diversity screen. (A) Initial hits were validated by demonstrating dose-dependent induction of the ISG54-luciferase reporter (left), absence of nonspecific promoter induction (β-actin-LUC, middle) and absence of cytotoxicity in multiple cell types (MTS assay, right). (B) Antiviral characterization measured inhibition of HCV focus formation (left) and viral RNA production in the supernatant (right) of Huh7 cells infected with a synthetic JFH-1 HCV 2A virus in combination with pre- or post-infection drug treatment. (C) Influenza studies characterized viral nucleoprotein production by ELISA (left) or Western blot (right) in drug-treated MRC5 cells infected with A/WSN/33 virus in comparison to control concentrations of IFN α-2a (Intron A, middle).

FIG. 14 shows IRF-3 nuclear translocation. IRF-3 (left panels) was examined in Huh7 cells 24 hours after treatment with KIN300, Sendai virus (positive control), or a negative control compound (10 μM) that did not induce ISG expression. IRF-3 was detected with rabbit polyclonal serum and a DyLight 488 secondary antibody (green) and nuclei were detected by Hoescht staining (blue). Poly (A) binding protein (right panels) was examined as a negative control using a monoclonal antibody and Dylight 488 (green).

### DETAILED DESCRIPTION

The present disclosure provides methods to identify compounds that shift the focus of viral treatments away from the targeting of viral proteins to the development of drugs that target and enhance the host (patient's) innate antiviral response. Such compounds and methods are likely to be more effective, less susceptible to the emergence of viral resistance, cause fewer side effects and be effective against a range of different viruses (1).

The RIG-I pathway is intimately involved in regulating the innate immune response to RNA virus infections. RIG-I is a cytosolic pathogen recognition receptor that is essential for triggering immunity to a wide range of RNA viruses (5-8). RIG-I is a double-stranded RNA helicase that binds to motifs within the RNA virus genome characterized by homopolymeric stretches of uridine or polymeric U/A motifs (9). Binding to RNA induces a conformation change that relieves RIG-I signaling repression by an autologous repressor domain, thus allowing RIG-I to signal downstream through its tandem caspase activation and recruitment domains (CARDs) (4). RIG-I signaling is dependent upon its NTPase activity, but does not require the helicase domain (10, 11). RIG-I signaling is silent in resting cells, and the repressor domain serves as the on-off switch that governs signaling in response to virus infection (8).

RIG-I signaling is transduced through IPS-1 (also known as Cardif, MAVs, and VISA), an essential adaptor protein that resides in the outer mitochondrial membrane (12-15). IPS-1 recruits a macromolecular signaling complex that stimulates the downstream activation of IRF-3, a transcription factor that induces the expression of type I interferons (IFNs) and virus-responsive genes that control infection (16). Compounds that trigger RIG-I signaling directly or through modulation of RIG-I pathway components, including IRF-3, present attractive therapeutic applications as antivirals or immune modulators.

A high-throughput screening approach was used to identify compounds that modulate the RIG-I pathway, a key regulator of the cellular innate immune response to RNA virus infection. In particular embodiments, validated RIG-I agonist lead compounds were demonstrated to specifically activate interferon regulatory factor-3 (IRF-3). In additional embodiments they exhibit one or more of the following: they induce the expression of interferon-stimulated genes (ISGs), have low cytotoxicity in cell-based assays, are suitable for analog development and QSAR studies, have drug-like physiochemical properties, and have antiviral activity against influenza A virus and/or hepatitis C virus (HCV). In certain embodiments, the compounds exhibit all of these characteristics. As discussed below, these compounds represent a new class of potential antiviral therapeutics. Although the disclosure is not bound by a specific mechanism of action of the compounds in vivo, the compounds are selected for their modulation of the RIG-I pathway. In certain embodiments, the modulation is activation of the RIG-I pathway.

Antiviral and mechanistic actions of lead compounds were used to identify a list of validated compounds suitable for optimization and pharmaceutical development experiments focused on HCV, influenza virus, and West Nile virus. Lead compounds disclosed herein function to, one or more of, decrease viral protein, viral RNA, and infectious virus in cell culture models of HCV and/or influenza virus.

Many RNA viruses share biochemical, regulatory, and signaling pathways. These viruses include but are not limited to influenza virus (including avian and swine isolates), Hepatitis C virus, West Nile virus, SARS-coronavirus, poliovirus, measles virus, Dengue virus, yellow fever virus, tick-borne encephalitis virus, Japanese encephalitis virus, St. Louis encephalitis virus, Murray Valley virus, Powassan virus, Rocio virus, Iouping-ill virus, Banzi virus, Ilheus virus, Kokobera virus, Kunjin virus, Alfuy virus, bovine diarrhea virus, and the Kyasanur forest disease virus. The methods described herein can be used to identify compounds that can be used to treat these viruses.

Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae. The compounds and methods disclosed herein can be used to treat viruses within these families of viruses as part of a pharmaceutically acceptable drug formulation. Other relevant virus families include, without limitation, Hepadnaviridae, Herpesviridae, Paramyxoviridae and Papillomaviridae.

The disclosure provides for a vaccine comprised of the compounds in combination with an antigen, for the purpose of preventing or treating disease in an animal including a vertebrate animal. As used herein, vaccines include compositions that act prophylactically or therapeutically to establish and/or enhance immunity of the host against disease and/or infection.

The disclosure provides for the use of the compounds as adjuvants. As used herein, adjuvant enhances, potentiates, prolongs, and/or accelerates the effects of another administered prophylactic and/or therapeutic agent including but not limited to a vaccine.

The disclosure also provides methods of identifying a therapeutic compound for preventing or inhibiting infection by a virus, wherein the therapeutic compound has the Structure KIN100 (isoflavone):

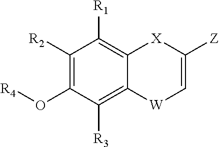

wherein R₁, R₂ and R₃ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO₂, OCF₃, CF₃, Br, Cl, F, 1-amidino, 2-amidino, alkylcarbonyl, morpholino, piperidinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR₅, SOR₆, SO₂R₇, CO₂R₈, COR₉, CONR₁₀R₁₁, CSNR₁₂R₁₃, SOₙNR₁₄R₁₅,
R₄ (independently) is H, lower alkyl, aryl, alkenyl, alkynyl, alkylaryl, arylalkyl, alkoxyalkylaryl, alkylamino, arylamino, heteroalkyl, heteroaryl, cyclic heteroalkyl, acyl, alkylsulfonyl, arylsulfonyl and heterocyclicalkylalkyl,

### W is O or NH,

### X is C═O, S═O or SO₂, and

Z is alkyl substituted alkyl, aryl, substituted aryl, heteroalkyl, heteroaryl, substituted heteroaryl, arylalkyl, heteroaryl alkyl.

Exemplary compounds include:

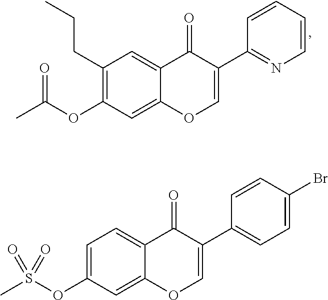 [SMILES: CCCc1cc2c(=O)c(-c3ccccn3)coc2cc1OC(C)=O ; CS(=O)(=O)Oc1ccc2c(=O)c(-c3ccc(Br)cc3)coc2c1]

The disclosure also provides methods of identifying a therapeutic compound for preventing or inhibiting infection by a virus, wherein the therapeutic compound has the structure KIN 200 (dihydrochalcone):

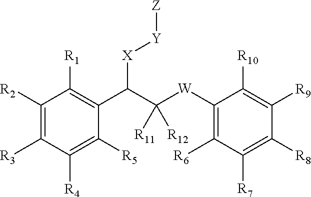

wherein R₁, R₂, R₃, R₄, R₅, R₆, R₇, R₈, R₉, R₁₀ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO₂, OCF₃, CF₃, Br, Cl, F, 1-amidino, 2-amidino, alkylcarbonyl, morpholino, piperidinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR₁₁, SOR₁₂, SO₂R₁₃, CO₂R₁₄, COR₁₅, CONR₁₆R₁₇, CSNR₁₈R₁₉, SOₙNR₂₀R₂₁,

### W is C═O, C═O(NH₂), S═O, SO₂, SO₂NH₂,

### X is S, O, NH, CR₂₂R₂₃, CR₂₄R₂₅CR₂₆R₂₇,

Y is lower alkyl, aryl, alkenyl, alkynyl, alkylaryl, arylalkyl, heteroalkyl, heteroaryl, or cyclic heteroalkyl,
Z is OH, NR₂₈R₂₉, NR₃₀CO₂R₃₁, NR₃₂(C═O)NR₃₃R₃₄, CO₂H, CO₂R₃₅, CONH₂, CONR₃₆R₃₇, C═O(R₃₈), 1-amidine, 2-amidine, guanidine, N-cyanoamidine, N-cyanoguanidine and tetrazole, and
R₁₁ through R₃₈ (independently) are H, lower alkyl, aryl, alkenyl, alkynyl, alkylaryl, arylalkyl, heteroalkyl, heteroaryl and cyclic heteroalkyl.

An exemplary compound includes:

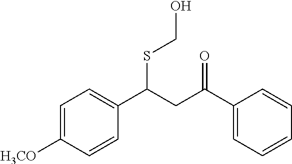 [SMILES: COc1ccc(C(CC(=O)c2ccccc2)SCO)cc1]

The disclosure also provides methods of identifying a therapeutic compound for preventing or inhibiting infection by a virus, wherein the therapeutic compound has the structure KIN 300A (thiazolidin-4-one 2-thione):

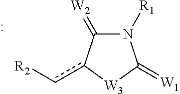

wherein W₁, W₂, W₃ (independently) are O, S, NH, NR₁; and
R₁, R₂ (independently, substituted or unsubstituted) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, heteroalkylaryl or acyl.

The disclosure also provides methods of identifying a therapeutic compound for preventing or inhibiting infection by a virus, wherein the therapeutic compound has the structure KIN300B (thiazolidin-4-one 2-thione):

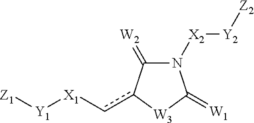

wherein W₁, W₂, W₃ (independently) are O, S, NH, NR₁;
X₁, X₂ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, heteroalkylaryl or acyl;
Y₁, Y₂ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, heteroalkylaryl or acyl;
Z₁, Z₂ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, heteroalkylaryl, acyl, Z═OH, OR₁, NR₂R₃, NR₄CO₂R₅, NR₆(C═O)NR₇R₈, CO₂H, CO₂R₉, CONR₁₀R₁₁, -amidine, 2-amidine, guanidine, N-cyanoamidine, N-cyanoguanidine, tetrazole CS(OR₁₂), SO₂R₁₃, COR₁₄, CONR₁₅R₁₆, SO₂NR₁₇R₁₈, O(C═O)NR₁₉;
R₁, R₂ (independently, substituted or unsubstituted) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, heteroalkylaryl or acyl.

An exemplary compound includes:

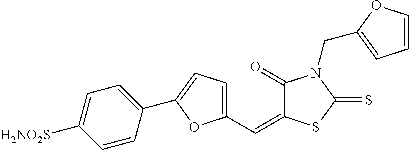 [SMILES: NS(=O)(=O)c1ccc(-c2ccc(/C=C3/SC(=S)N(Cc4ccco4)C3=O)o2)cc1]

The disclosure also provides methods of identifying a therapeutic compound for preventing or inhibiting infection by a virus, wherein the therapeutic compound has the structure KIN 400 (diarylpyridine):

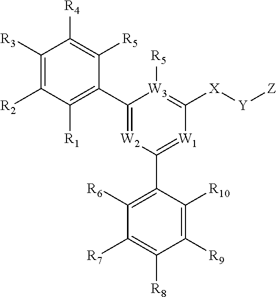

wherein R₁, R₂, R₃, R₄, R₅, R₆, R₇, R₈, R₉, R₁₀ (independently) are H, alkyl, cycloalkyl, aryl, alkyl aryl, Br, Cl, F, OH, OR₅, NH₂, NR₁₁R₁₂, NO₂, SR₁₃, SOR₁₄, SO₂R₁₅, COR₁₆, CONR₁₇R₁₈, SO₂NR₁₉R₂₀, and NR₂₁ or SO₂R₂₂;
W₁, W₂, W₃ (independently) are N, CH, CR₂₃;
X is S, NH, NR₂₄, O, (CR₂₅R₂₆)ₙ₁;
n₁ is 0 to 8;
Y is S, NH, NR₂₇, O, (CR₂₈R₂₉)ₙ₂;
n₂ is 0 to 8;
Z is CH₂OH, CH₂NH₂, CH₂NR₃OR₃₁, CO₂H, CO₂R₃₂, CONH₂, CONR₃₃R₃₄, C═O(R₃₅), and tetrazole; and
R₁₀ through R₃₅ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, or
when taken together form rings including but not limited to piperidine, piperazine, oxetane, pyrrolidine, pyran, dioxane or methylene dioxane.

An exemplary compound includes:

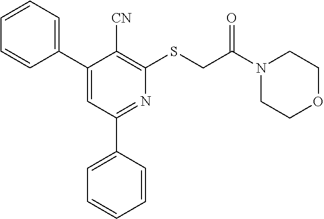 [SMILES: N#Cc1c(-c2ccccc2)cc(-c2ccccc2)nc1SCC(=O)N1CCOCC1]

The disclosure also provides methods of identifying a therapeutic compound for preventing or inhibiting infection by a virus, wherein the therapeutic compound has the structure KIN500 (N,N′-polyalkylated uracil):

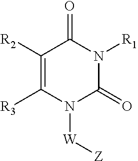

wherein R₁ is H, alkyl, cycloalkyl, aryl, alkylaryl, arylalkyl, heteroalkyl, heterocyclic aryl, cyclic cyclic heteroalkyl; SO₂R₁, or SO₂NR₂R₃;
R₂, R₃ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO₂, OCF₃, CF₃, Br, Cl, F, 1-amidino, 2-amidino, alkylcarbonyl, morpholino, piperidinyl, dioxanyl, pyranyl, heteroaryl, furanyl, thiophenyl, tetrazole, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR₄, SOR₅, SO₂R₆, CO₂R₇, COR_{S}, CONR₉R₁₀, CSNR₁₁R₁₂, SOₙNR₁₃R₁₄; or R₂, R₃ when taken together form an aromatic ring such as phenyl, thiophene, furan, imidazole, thiazole, isothiazole, oxazole, isoxazole, pyrrole or diazole, benzthiazole, benzofuran, benzoxazole, benzisoxazole, benzthiophene, benzimidazole, benzimidazole, benzopyran, benzodioxane;
n is 1 or 2;
R₄ through R₁₄ (independently) are H, lower alkyl, aryl, alkenyl, alkynyl, alkylaryl, arylalkyl, alkoxy, aryloxy, arylalkoxy, alkoxyalkylaryl, heteroalkyl, heteroaryl, cyclic heteroalkyl;
W is H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, C═O, O, S, NH, NR₁₅, (CR₁₆R₁₇)ₙ, (C═O)R₁₈; and
Z is OH, OR₁₉, NR₂₀R₂₁, NR₂₂CO₂R₂₃, NR₂₄(C═O)NR₂₅R₂₆, CO₂H, CO₂R₂₇, CONH₂, CONR₂₈R₂₉, 1-amidine, 2-amidine, guanidine, N-cyanoamidine, N-cyanoguanidine and tetrazole, CO₂H, CS(OR₃₀), SO₂R₃₁, COR₃₂, CONR₃₃R₃₄, SO₂NR₃₅R₃₆, and NR₃₇ or SO₂R₃₈. An exemplary compound includes:

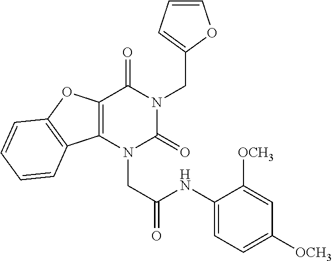 [SMILES: COc1ccc(NC(=O)Cn2c(=O)n(Cc3ccco3)c(=O)c3oc4ccccc4c32)c(OC)c1]

The disclosure also provides methods of identifying a therapeutic compound for preventing or inhibiting infection by a virus, wherein the therapeutic compound has the structure KIN600 (diarylsulfonamide):

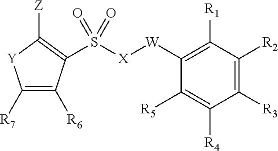

wherein R₁, R₂, R₃, R₄, R₅, R₆, R₇ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO₂, OCF₃, CF₃, Br, Cl, F, 1-amidino, 2-amidino, alkylcarbonyl, morpholino, piperidinyl, dioxanyl, pyranyl, heteroaryl, furanyl, thiophenyl, tetrazole, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR₅, SOR₆, SO₂R₇, CO₂R₈, COR₉, CONR₁₀R₁₁, CSNR₁₂R₁₃, SOₙNR₁₄R₁₅;

### W is O, (CR₈R₉)n;

R₈R₉ (independently) are H, lower alkyl, aryl, alkenyl and alkynyl;
n is 0-7;

### X is NH, NR₁₀;

### Y is O, NH, NR₁₁, S, CH═CH, CR₁₂═CR₁₃;

Z is CH₂OH, CH₂NH₂, CH₂NR₁₄R₁₅, CO₂H, CO₂R₁₆, CONH₂, CONR₁₇R₁₈ and tetrazole; and
R₁₀, R₁₁, R₁₂, R₁₃, R₁₄, R₁₅, R₁₆, R₁₇, R₁₈ (independently) are H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, heteroalkyl, heteroaryl, or cyclic heteroalkyl.

An exemplary compound includes:

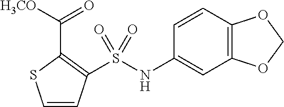 [SMILES: COC(=O)c1sccc1S(=O)(=O)Nc1ccc2c(c1)OCO2]

The disclosure also provides methods of identifying a therapeutic compound for preventing or inhibiting infection by a virus, wherein the therapeutic compound has the structure KIN700 (imidate thioamide):

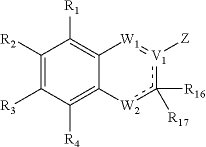

wherein, R₁, R₂, R₃ and R₄ (independently) are H, lower alkyl, aryl, alkenyl, alkynyl, alkylaryl, arylalkyl, alkoxy, aryloxy, arylalkoxy, alkoxyalkylaryl, alkylamino, arylamino, NH₂, OH, CN, NO₂, OCF₃, CF₃, Br, Cl, F, 1-amidino, 2-amidino, alkylcarbonyl, morpholino, piperidinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR₅, SOR₆, SO₂R₇, CO₂R₈, COR₉, CONR₁₀R₁₁, CSNR₁₂R₁₃, SOₙNR₁₄R₁₅
wherein R₅, R₆, R₇, R₈, R₉, R₁₀, R₁₁, R₁₂, R₁₃, R₁₄ and R₁₅ (independently)=H, lower alkyl, aryl, alkenyl, alkynyl, alkylaryl, arylalkyl heteroalkyl, heteroalkylaryl, heteroalkylarylalkyl, heteroaryl, heteroarylalkyl, cyclicalkyl, cyclicalkylaryl, heterocyclicalkyl, heterocyclicalkylalkyl; and
wherein R₁₆, R₁₇ (independently) are H, lower alkyl, aryl, alkenyl, alkynyl, alkylaryl, arylalkyl, cyclicalkyl, arylcyclicalkyl, heterocyclicalkyl, heterocyclicalkylalkyl, heteroalkyl, heteroalkylaryl, arylheteroalkyl, or heteroalkylarylalkyl; or
when taken together are alkylimine, arylimine, NR₁₈, CR₁₈, spiroalkyl, spiroheteroalkyl, NH₂, OH, CN, NO₂, OCF₃, CF₃, Br, Cl, F;
W₁, W₂ (independently) are CH, CR₁₉R₂₀, N, NH, NR₂₁, O, SO, SO₂;

### V₁ is C or N;

Z is CO₂R₂₂, COR₂₂, CONR₂₂R₂₃, C═S(NR₂₂R₂₃), SOₙR₂₂, 1-amidino, 2-amidino, tetrazole, hydroxamic acid, ureido, thioureido, carbamoyl, N-cyanoamidine, N-sulfonamido amidine, NH₂, OH, CN, NO₂, OCF₃, CF₃, Br, Cl, F, 1-amidine, 2-amidine, alkylcarbonyl, morpholine, piperidine, dioxane, pyran, heteroaryl, furanyl, thiophenyl, tetrazole, thiazole, isothiazole, imidazole, thiadiazole, thiadiazole S-oxide, thiadiazole S,S-dioxide, pyrazole, oxazole, isoxazole, pyridinyl, pyrimidinyl, quinazoline, quinoline, isoquinoline, SR₂₂, SOR₂₂, SO₂R₂₂, CO₂R₂₂, COR₂₂, CONR₂₂R₂₃, CSNR₂₂R₂₃, SOₙNR₂₂R₂₃;
wherein R₁₈, R₁₉, R₂₀, R₂₁, R₂₂, R₂₃, R₂₄, R₂₅ (independently) (independently) are H, lower alkyl, aryl, alkenyl, alkynyl, alkylaryl, arylalkyl heteroalkyl, heteroalkylaryl, heteroalkylarylalkyl, heteroaryl, heteroarylalkyl, cyclicalkyl, cyclicalkylaryl, heterocyclicalkyl, heterocyclicalkylalkyl.

An exemplary compound includes:

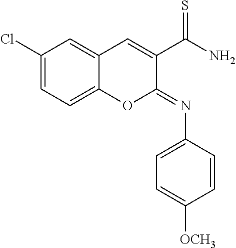 [SMILES: COc1ccc(/N=c2\oc3ccc(Cl)cc3cc2C(N)=S)cc1]

As used herein, either alone or in combination, the terms “alkyloxy” or “alkoxy” refer to a functional group comprising an alkyl ether group. Examples of alkoxys include, without limitation, methoxy, ethoxy, n-propoxy, isopropoxy, n-butoxy, iso-butoxy, sec-butoxy, tert-butoxy, and the like.

The terms “alkyl”, “alkenyl”, and “alkynyl” refer to substituted and unsubstituted alkyls, alkenyls and alkynyls. The term “alkyl” refers to a functional group comprising a straight-chain or branched-chain hydrocarbon containing from 1 to 20 carbon atoms linked exclusively by single bonds and not having any cyclic structure. An alkyl group may be optionally substituted as defined herein. Examples of alkyl groups includes, without limitation methyl, ethyl, n-propyl, isopropyl, n-butyl, isobutyl, sec-butyl, tert-butyl, pentyl, iso-amyl, hexyl, heptyl, octyl, noyl, decyl, undecyl, dodecyl tridecyl, tetradecyl, pentadecyl, hexadecyl, heptadecyl, octadecyl, nonadecyl, eicosyl, and the like.

Substituted alkyls, alkenyls and alkynyls refers to alkyls, alkenyls and alkynyls substituted with one to five substituents from the group including H, lower alkyl, aryl, alkenyl, alkynyl, arylalkyl, alkoxy, aryloxy, arylalkoxy, alkoxyalkylaryl, alkylamino, arylamino, NH2, OH, CN, NO2, OCF3, CF3, F, 1-amidine, 2-amidine, alkylcarbonyl, morpholinyl, piperidinyl, dioxanyl, pyranyl, heteroaryl, furanyl, thiophenyl, tetrazolo, thiazolyl, isothiazolyl, imidazolyl, thiadiazolyl, thiadiazole S-oxide, thiadiazole S,S-dioxide, pyrazolo, oxazolyl, isoxazolyl, pyridinyl, pyrimidinyl, quinolinyl, isoquinolinyl, SR, SOR, SO2R, CO2R, COR, CONR′R″, CSNR′R″, SOnNR′R″.

As used herein, either alone or in combination, the term “alkynyl” refers to a functional group comprising a straight-chain or branched-chain hydrocarbon containing from 2 to 20 carbon atoms and having one or more carbon-carbon triple bonds and not having any cyclic structure. An alkynyl group may be optionally substituted as defined herein. Examples of alkynyl groups include, without limitation, ethynyl, propynyl, hydroxypropynyl, butynyl, butyn-1-yl, butyn-2-yl, 3-methylbutyn-1-yl, pentynyl, pentyn-1-yl, hexynyl, hexyn-2-yl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl, eicosynyl, and the like.

The term “alkylene,” as used herein, alone or in combination, refers to a saturated aliphatic group derived from a straight or branched chain saturated hydrocarbon attached at two or more positions, such as methylene (—C2-). Unless otherwise specified, the term “alkyl” may include “alkylene” groups.

As used herein, either alone or in combination, the term “alkylcarbonyl” or “alkanoyl” refers to a functional group comprising an alkyl group attached to the parent molecular moiety through a carbonyl group. Examples of alkylcarbonyl groups include, without limitation, methylcarbonyl, ethylcarbonyl, and the like.

The term “alkynylene” refers to a carbon-carbon triple bond attached at two positions such as ethynylene (—C:::C—, —C═C—). Unless otherwise specified, the term “alkynyl” may include “alkynylene” groups.

As used herein, either alone or in combination, the term “aryl”, “hydrocarbyl aryl”, or “aryl hydrocarbon” refers to a functional group comprising a substituted or unsubstituted aromatic hydrocarbon with a conjugated cyclic molecular ring structure of 3 to 12 carbon atoms. An aryl group can be monocyclic, bicyclic or polycyclic, and may optionally include one to three additional ring structures, such as, e.g., a cycloalkyl, a cycloalkenyl, a heterocycloalkyl, a heterocycloalkenyl, or a heteroaryl. The term “aryl” includes, without limitation, phenyl(benzenyl), thiophenyl, indolyl, naphthyl, totyl, xylyl, anthracenyl, phenanthryl, azulenyl, biphenyl, naphthalenyl, 1-mMethylnaphthalenyl, acenaphthenyl, acenaphthylenyl, anthracenyl, fluorenyl, phenalenyl, phenanthrenyl, benzo[a]anthracenyl, benzo[c]phenanthrenyl, chrysenyl, fluoranthenyl, pyrenyl, tetracenyl(naphthacenyl), triphenylenyl, anthanthrenyl, benzopyrenyl, benzo[a]pyrenyl, benzo[e]fluoranthenyl, benzo[ghi]perylenyl, benzo[j]fluoranthenyl, benzo[k]fluoranthenyl, corannulenyl, coronenyl, dicoronylenyl, helicenyl, heptacenyl, hexacenyl, ovalenyl, pentacenyl, picenyl, perylenyl, and tetraphenylenyl. Substituted aryl refers to aryls substituted with one to five substituents from the group including H, lower alkyl, aryl, alkenyl, alkynyl, arylalkyl, alkoxy, aryloxy, arylalkoxy, alkoxyalkylaryl, alkylamino, arylamino, NH₂, OH, CN, NO₂, OCF₃, CF₃, Br, Cl, F, 1-amidino, 2-amidino, alkylcarbonyl, morpholino, piperidinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR, SOR, SO₂R, CO₂R, COR, CONRR, CSNRR, SOnNRR.

As used herein, either alone or in combination, the term “lower aryl” refers to a functional group comprising a substituted or unsubstituted aromatic hydrocarbon with a conjugated cyclic molecular ring structure of 3 to 6 carbon atoms. Examples of lower aryl groups include, without limitation, phenyl and naphthyl.

As used herein, either alone or in combination, the term “carboxyl” or “carboxy” refers to the functional group —C(═O)OH or the corresponding “carboxylate” anion —C(═O)O—. Examples include, without limitation, formic acid, acetic acid, oxalic acid, benzoic acid. An “O-carboxyl” group refers to a carboxyl group having the general formula RCOO, wherein R is an organic moeity or group. A “C-carboxyl” group refers to a carboxyl group having the general formula COOR, wherein R is an organic moeity or group.

As used herein, either alone or in combination, the term “cycloalkyl”, “carbocyclicalkyl”, and “carbocyclealkyl” refers to a functional group comprising a substituted or unsubstituted non-aromatic hydrocarbon with a non-conjugated cyclic molecular ring structure of 3 to 12 carbon atoms linked exclusively with carbon-carbon single bonds in the carbon ring structure. A cycloalkyl group can be monocyclic, bicyclic or polycyclic, and may optionally include one to three additional ring structures, such as, e.g., an aryl, a heteroaryl, a cycloalkenyl, a heterocycloalkyl, or a heterocycloalkenyl.

As used herein, either alone or in combination, the term “lower cycloalkyl” refers to a functional group comprising a monocyclic substituted or unsubstituted non-aromatic hydrocarbon with a non-conjugated cyclic molecular ring structure of 3 to 6 carbon atoms linked exclusively with carbon-carbon single bonds in the carbon ring structure. Examples of lower cycloalkyl groups include, without limitation, cyclopropyl, cyclobutyl, cyclopentyl, and cyclohexyl.

As used herein, the term “functional group” refers to a specific group of atoms within a molecule that are responsible for the characteristic chemical reactions of those molecules.

As used herein, either alone or in combination, the term “heteroalkyl” refers to a functional group comprising a straight-chain or branched-chain hydrocarbon containing from 1 to 20 atoms linked exclusively by single bonds, where at least one atom in the chain is a carbon and at least one atom in the chain is O, S, N, or any combination thereof. The heteroalkyl group can be fully saturated or contain from 1 to 3 degrees of unsaturation. The non-carbon atoms can be at any interior position of the heteroalkyl group, and up to two non-carbon atoms may be consecutive, such as, e.g., —CH2-NH—OCH3. In addition, the non-carbon atoms may optionally be oxidized and the nitrogen may optionally be quaternized.

As used herein, either alone or in combination, the term “heteroaryl” refers to a functional group comprising a substituted or unsubstituted aromatic hydrocarbon with a conjugated cyclic molecular ring structure of 3 to 12 atoms, where at least one atom in the ring structure is a carbon and at least one atom in the ring structure is O, S, N, or any combination thereof. A heteroaryl group can be monocyclic, bicyclic or polycyclic, and may optionally include one to three additional ring structures, such as, e.g., an aryl, a cycloalkyl, a cycloalkenyl, a heterocycloalkyl, or a heterocycloalkenyl. Examples of heteroaryl groups include, without limitation, acridinyl, benzidolyl, benzimidazolyl, benzisoxazolyl, benzodioxinyl, dihydrobenzodioxinyl, benzodioxolyl, 1,3-benzodioxolyl, benzofuryl, benzoisoxazolyl, benzopyranyl, benzothiophenyl, benzo[c]thiophenyl, benzotriazolyl, benzoxadiazolyl, benzoxazolyl, benzothiadiazolyl, benzothiazolyl, benzothienyl, carbazolyl, chromonyl, cinnolinyl, dihydrocinnolinyl, coumarinyl, dibenzofuranyl, furopyridinyl, furyl, indolizinyl, indolyl, dihydroindolyl, imidazolyl, indazolyl, isobenzofuryl, isoindolyl, isoindolinyl, dihydroisoindolyl, isoquinolyl, dihydroisoquinolinyl, isoxazolyl, isothiazolyl, oxazolyl, oxadiazolyl, phenanthrolinyl, phenanthridinyl, purinyl, pyranyl, pyrazinyl, pyrazolyl, pyridyl, pyrimidinyl, pyridazinyl, pyrrolinyl, pyrrolyl, pyrrolopyridinyl, quinolyl, quinoxalinyl, quinazolinyl, tetrahydroquinolinyl, tetrazolopyridazinyl, tetrahydroisoquinolinyl, thiophenyl, thiazolyl, thiadiazolyl, thienopyridinyl, thienyl, thiophenyl, triazolyl, xanthenyl, and the like.

As used herein, either alone or in combination, the term “lower heteroaryl” refers to a functional group comprising a monocyclic or bicyclic, substituted or unsubstituted aromatic hydrocarbon with a conjugated cyclic molecular ring structure of 3 to 6 atoms, where at least one atom in the ring structure is a carbon and at least one atom in the ring structure is O, S, N, or any combination thereof.

As used herein, either alone or in combination, the term “hydroxy” refers to the functional group hydroxyl (—OH).

As used herein, either alone or in combination, the term “oxo” refers to the functional group ═O.

As used herein, the term “vertebrate” includes all living vertebrates such as, without limitation, mammals, humans, birds, dogs, cats, livestock, farm animals, free-range herds, etc.

As used herein, a “pharmaceutical composition” comprises at least one compound disclosed herein together with one or more pharmaceutically acceptable carriers, excipients or diluents, as appropriate for the chosen mode of administration.

The pharmaceutical compositions can be made up in, without limitation, a solid form (including granules, powders or suppositories) or in a liquid form (e.g., solutions, suspensions, or emulsions). The pharmaceutical compositions can be subjected to conventional pharmaceutical operations such as sterilization and/or can contain conventional adjuvants, such as preservatives, stabilizers, wetting agents, emulsifiers, buffers etc.

Solid dosage forms for oral administration can include capsules, tablets, pills, powders, and granules. In such solid dosage forms, the active compound can be admixed with at least one inert diluent such as sucrose, lactose, or starch. Such dosage forms can also comprise, as in normal practice, additional substances other than inert diluents, e.g., lubricating agents such as magnesium stearate. In the case of capsules, tablets, and pills, the dosage forms can also comprise buffering agents. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting, sweetening, flavoring, and perfuming agents. The pharmaceutical composition can contain more than one embodiment of the present invention. Preparations for oral administration can be suitably formulated to give controlled release of the active compound.

For buccal administration the compositions can take the form of tablets or lozenges formulated in conventional manner.

The compounds can be formulated for parenteral administration by injection e.g. by bolus injection or infusion. Formulations for injection can be presented in unit dosage form, e.g. in glass ampoule or multi dose containers, e.g. glass vials. The compositions for injection can take such forms as suspensions, solutions or emulsions in oily or aqueous vehicles, and can contain formulatory agents such as suspending, stabilising, preserving and/or dispersing agents. Alternatively, the active ingredient can be in powder form for constitution with a suitable vehicle, e.g. sterile pyrogen-free water, before use.

In addition to the formulations described above, the compounds can also be formulated as a depot preparation. Such long acting formulations can be administered by implantation or by intramuscular injection.

For nasal or pulmonary administration or any other administration by inhalation, the compounds for use according to the present invention are conveniently delivered in the form of an aerosol spray presentation for pressurized packs or a nebulizer, with the use of suitable propellant, e.g. dichlorodifluoromethane, trichlorofluoromethane, dichlorotetrafluoroethane, carbon dioxide or other suitable gas or mixture of gases.

The compounds and methods disclosed herein can be additive or synergistic with other therapies currently in development or use. For example, ribavirin and interferon-α (IFN-α) provide an effective treatment for HCV infection when used in combination. Their efficacy in combination can exceed the efficacy of either drug product when used alone. The compositions of the disclosure can be administered alone or in combination or conjunction with IFN-α, ribavirin and/or a variety of small molecules that are being developed against both viral targets (viral proteases, viral polymerase, assembly of viral replication complexes) and host targets (host proteases required for viral processing, host kinases required for phosphorylation of viral targets such as NS5A, and inhibitors of host factors required to efficiently utilize the viral internal ribosome entry site, or IRES).

The compounds and methods disclosed herein could be used in combination or conjunction with, without limitation, adamantane inhibitors, neuraminidase inhibitors, alpha interferons, non-nucleoside or nucleoside polymerase inhibitors, NS5A inhibitors, antihistamines, protease inhibitors, helicase inhibitors, P7 inhibitors, entry inhibitors, IRES inhibitors, immune stimulators, HCV replication inhibitors, cyclophilin A inhibitors, A3 adenosine agonists, and microRNA suppressors.

Cytokines that could be administered in combination or conjunction with the compounds and methods disclosed herein include, without limitation, IL-2, IL-12, IL-23, IL-27, or IFN-γ. New HCV drugs that are or will be available for potential administration in combination or conjunction with the compounds and methods disclosed herein include, without limitation, ACH-1625 (Achillion); Glycosylated interferon (Alios Biopharma); ANA598, ANA773 (Anadys Pharm); ATI-0810 (Arisyn Therapeutics); AVL-181 (Avila Therapeutics); LOCTERON® (Biolex); CTS-1027 (Conatus); SD-101 (Dynavax Technologies); Clemizole (Eiger Biopharmaceuticals); GS-9190 (Gilead Sciences); GI-5005 (GlobalImmune BioPharma); Resiquimod/R-848 (Graceway Pharmaceuticals); Albinterferon alpha-2b (Human Genome Sciences); IDX-184, IDX-320, IDX-375 (Idenix); IMO-2125 (Idera Pharmaceuticals); INX-189 (Inhibitex); ITCA-638 (Intarcia Therapeutics); ITMN-191/RG7227 (Intermune); ITX-5061, ITX-4520 (iTherx Pharmaceuticals); MB11362 (Metabasis Therapeutics); Bavituximab (Peregrine Pharmaceuticals); PSI-7977, RG7128, PSI-938 (Pharmasset); PHX1766 (Phenomix); Nitazoxanide/ALINIA® (Romark Laboratories); SP-30 (Samaritan Pharmaceuticals); SCV-07 (SciClone); SCY-635 (Scynexis); TT-033 (Tacere Therapeutics); Viramidine/taribavirin (Valeant Pharmaceuticals); Telaprevir, VCH-759, VCH-916, VCH-222, VX-500, VX-813 (Vertex Pharmaceuticals); and PEG-INF Lambda (Zymogenetics).

New influenza and West Nile virus drugs that are or will be available for potential administration in combination or conjunction with the compounds and methods disclosed herein include, without limitation, neuraminidase inhibitors (Peramivir, Laninamivir); triple therapy—neuraminidase inhibitors ribavirin, amantadine (ADS-8902); polymerase inhibitors (Favipiravir); reverse transcriptase inhibitor (ANX-201); inhaled chitosan (ANX-211); entry/binding inhibitors (Binding Site Mimetic, Flucide); entry inhibitor, (Fludase); fusion inhibitor, (MGAWN1 for West Nile); host cell inhibitors (lantibiotics); cleavage of RNA genome (RNAi, RNAse L); immune stimulators (Interferon, Alferon-LDO; Neurokinin) agonist, Homspera, Interferon Alferon N for West Nile); and TG21.

Other drugs for treatment of influenza and/or hepatitis that are available for potential administration in combination or conjunction with the compounds and methods disclosed herein include, without limitation:

TABLE 1
Hepatitis and influenza drugs

| Branded Name | Generic Name | Approved Indications |
|---|---|---|
| Pegasys | PEGinterferon alfa-2a | Hepatitis C, Hepatitis B |
| Peg-Intron | PEGinterferon alfa-2b | Hepatitis C |
| Copegus | Ribavirin | Hepatitis C |
| Rebetol | Ribavirin | Hepatitis C |
| — | Ribavirin | Hepatitis C |
| Tamiflu | Oseltamivir | Influenza A, B, C |
| Relenza | Zanamivir | Influenza A, B, C |
| — | Amantadine | Influenza A |
| — | Rimantadine | Influenza A |

These agents can be incorporated as part of the same pharmaceutical composition or can be administered separately from the compounds of the disclosure, either concurrently or in accordance with another treatment schedule. In addition, the compounds or compositions of the disclosure can be used as an adjuvant to other therapies.

The compounds and methods disclosed herein can be additive or synergistic with other compounds and methods to enable vaccine development. By virtue of their antiviral and immune enhancing properties, the compounds can be used to affect a prophylactic or therapeutic vaccination. The compounds need not be administered simultaneously or in combination with other vaccine components to be effective. The vaccine applications of the compounds are not limited to the prevention or treatment of virus infection but can encompass all therapeutic and prophylactic vaccine applications due to the general nature of the immune response elicited by the compounds.

As is understood by one of ordinary skill in the art, vaccines can be against viruses, bacterial infections, cancers, etc. and can include one or more of, without limitation, a live attenuated vaccine (LAIV), an inactivated vaccine (IIV; killed virus vaccine), a subunit (split vaccine); a sub-virion vaccine; a purified protein vaccine; or a DNA vaccine. Appropriate adjuvants include one or more of, without limitation, water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs such as N-acetyl-nor-muranyl-L-alanyl-D-isoglutamine, N-acetyl-muranyl-(6-O-stearoyl)-L-alanyl-D-isoglutamine or N-Glycol-muranyl-LalphaAbu-D-isoglutamine (Ciba-Geigy Ltd.).

The pharmaceutical composition comprising a compound of the disclosure can be formulated in a variety of forms, e.g., as a liquid, gel, lyophilized, or as a compressed solid. The preferred form will depend upon the particular indication being treated and will be apparent to one of ordinary skill in the art. In one embodiment, the disclosed RIG-I agonists include formulations for oral delivery that can be small-molecule drugs that employ straightforward medicinal chemistry processes.

The administration of the formulations of the present disclosure can be performed in a variety of ways, including, but not limited to, orally, subcutaneously, intravenously, intracerebrally, intranasally, transdermally, intraperitoneally, intramuscularly, intrapulmonary, intrathecally, vaginally, rectally, intraocularly, or in any other acceptable manner. The formulations can be administered continuously by infusion, although bolus injection is acceptable, using techniques well known in the art, such as pumps (e.g., subcutaneous osmotic pumps) or implantation. In some instances the formulations can be directly applied as a solution or spray.

An example of a pharmaceutical composition is a solution designed for parenteral administration. Although in many cases pharmaceutical solution formulations are provided in liquid form, appropriate for immediate use, such parenteral formulations can also be provided in frozen or in lyophilized form. In the former case, the composition must be thawed prior to use. The latter form is often used to enhance the stability of the active compound contained in the composition under a wider variety of storage conditions, as it is recognized by those of ordinary skill in the art that lyophilized preparations are generally more stable than their liquid counterparts. Such lyophilized preparations are reconstituted prior to use by the addition of one or more suitable pharmaceutically acceptable diluents such as, without limitation, sterile water for injection or sterile physiological saline solution.

Parenterals can be prepared for storage as lyophilized formulations or aqueous solutions by mixing, as appropriate, the compound having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives can be added to retard microbial growth, and are typically added in amounts of about 0.2%-1% (w/v). Suitable preservatives for use with the present disclosure include, without limitation, phenol, benzyl alcohol, meta-cresol, methyl paraben, propyl paraben, octadecyldimethylbenzyl ammonium chloride, benzalkonium halides (e.g., benzalkonium chloride, bromide or iodide), hexamethonium chloride, alkyl parabens such as methyl or propyl paraben, catechol, resorcinol, cyclohexanol and 3-pentanol.

Isotonicifiers can be added to ensure isotonicity of liquid compositions and include, without limitation, polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, alpha-monothioglycerol and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active compound weight.

Additional miscellaneous excipients include bulking agents or fillers (e.g., starch), chelating agents (e.g., EDTA), antioxidants (e.g., ascorbic acid, methionine, vitamin E) and cosolvents.

The active ingredient can also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington, The Science and Practice of Pharmacy, 21^{st} Ed., published by Lippincott Williams & Wilkins, A Wolters Kluwer Company, 2005.

Parenteral formulations to be used for in vivo administration generally are sterile. This is readily accomplished, for example, by filtration through sterile filtration membranes.

Suitable examples of sustained-release preparations include semi-permeable matrices of solid hydrophobic polymers containing the compound or composition, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the PROLEASE® technology or LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release compounds for shorter time periods.

Oral administration of the compounds and compositions is one intended practice of the disclosure. For oral administration, the pharmaceutical composition can be in solid or liquid form, e.g., in the form of a capsule, tablet, powder, granule, suspension, emulsion or solution. The pharmaceutical composition is preferably made in the form of a dosage unit containing a given amount of the active ingredient. A suitable daily dose for a human or other vertebrate can vary widely depending on the condition of the patient and other factors, but can be determined by persons of ordinary skill in the art using routine methods.

In solid dosage forms, the active compound can be admixed with at least one inert diluent such as sucrose, lactose, or starch. Such dosage forms can also comprise, as is normal practice, additional substances, e.g., lubricating agents such as magnesium stearate. In the case of capsules, tablets and pills, the dosage forms can also comprise buffering agents. Tablets and pills can additionally be prepared with enteric coatings.

The compounds or compositions can be admixed with adjuvants such as lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, they can be dissolved in saline, water, polyethylene glycol, propylene glycol, ethanol, oils (such as corn oil, peanut oil, cottonseed oil or sesame oil), tragacanth gum, and/or various buffers. Other adjuvants and modes of administration are well known in the pharmaceutical art. The carrier or diluent can include time delay material, such as glyceryl monostearate or glyceryl distearate alone or with a wax, or other materials well known in the art.

The Examples below describe the optimization of the methods disclosed herein. The Examples below are included to demonstrate particular embodiments of the disclosure. It should be appreciated by those of ordinary skill in the art that the techniques disclosed in the Examples represent techniques and compositions discovered by the inventors to function well in the practice of the disclosure, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the disclosure.

The Examples provide in vitro methods for testing compounds for RIG-I agonist and/or anti-viral activity of the disclosure. Other in vitro virus infection models that can be used include but are not limited to flaviviruses such as bovine diarrheal virus, West Nile Virus, and GBV-C virus, other RNA viruses such as respiratory syncytial virus, and the HCV replicon systems (32). Any appropriate cultured cell competent for viral replication can be utilized as antiviral assays.

### EXAMPLES

In the Examples and this disclosure, TSA-A is the same compound as KIN200, TSA-B is the same compound as KIN100 and TSA-G is the same compound as KIN600.

### Example 1

Reporter Huh7 cell lines were developed to stably express firefly luciferase utilizing the ISG54 promoter cloned from genomic DNA. These cell lines are responsive to RIG-I mediated stimulus including Sendai virus infection as well as IFN treatment and are utilized to identify RIG-I agonists through high throughput screening (HTS) of a small molecule library. Induction of reporter cell lines was optimized for cell growth and assay conditions that are used in the HTS to obtain the most sensitive and reproducible results. Additionally, a control cell line that expresses Renilla luciferase using the actin promoter was developed as a negative control. The actin cell line is utilized in a counter screen to identify compounds that cause nonspecific changes in global gene expression.

Cloning of ISG54 and β-actin promoter constructs: Actin, ISG54 and ISG56 promoter sequences were amplified from stock genomic DNA using the following primers:

ISG54 For_Sac1:
(SEQ ID NO: 1)
GGGAGCTCCTCCGGAGGAAAAAGAGTCC
ISG54 Rev_EcoRV:
(SEQ ID NO: 2)
GGGATATCGCAGCTGCACTCTTGAGAAA
ISG56 For_Sac1:
(SEQ ID NO: 3)
GGGAGCTCATGGTTGCAGGTCTGCAGTT
ISG56 Rev_EcoRV:
(SEQ ID NO: 4)
GGGATATCTCTGGCTATTCTGTCTTGTGGA
Actin 5′ SacI:
(SEQ ID NO: 5)
GGGAGCTCCCCAAGGCGGCCAAC
Actin 3′ HindIII:
(SEQ ID NO: 6)
GGAAGCTTGGTCTCGGCGGTGGT

Sequence fragments were amplified using Platinum PCR reactions. The PCR fragments were purified, digested with SacI and EcoRV or HindIII and ligated into the Promega luciferase vectors. The actin promoter sequence was ligated into the pGL4.76 vector which contains a hygromycin selectable marker. ISG54 and ISG56 promoter sequences were ligated into the pGL4.17 vector that contains a puromycin selectable marker. Constructs were confirmed by sequencing and plasmid maps.

Production of stable cell lines: Huh7 cells were seeded in a 6-well plate at a density of 2.5×10⁵ cells per well and grown overnight under normal growth conditions prior to transfection. Cells were transfected with 2 μg of the appropriate vector DNA using Lipofectin and Plus Reagent from Invitrogen. Transfections were done using suggested reagent volumes and ratios provided in the Invitrogen protocol. Following transfection, cells were grown to confluency (24-48 hours) and each well was then split into two 10 cm dishes. Cells were grown 24 hours and then media was replaced with selective media containing the appropriate antibiotic. The optimal concentration of antibiotics for Huh7 cells was determined to be 400 μg/mL G418 (puromyocin) and 250 μg/mL hygromycin. Cells were grown in the presence of antibiotic until 80% of cells died under selective pressure and individual colonies appeared. Colonies that contained greater than 50 cells were trypsinized from the plate, transferred to a 96-well plate and grown in the presence of antibiotic (only 20-40% of the clones survive this phase). Surviving clones were grown and passaged when they reached 80% confluency under normal conditions but with media containing antibiotic. All stable cell line clones were frozen in liquid nitrogen and included in a cell bank.

Luciferase assays: Huh7 cells were grown under normal growth conditions and seeded in a 96-well plate at a density of 1×10⁴ cells per well and grown to 80% confluence (usually 20 hours). The positive control wells were infected with Sendai virus or treated with IFN at the designated concentration and incubated at 37 degrees for an additional 18-24 hours. Media was removed and cells were washed once with PBS. Passive lysis buffer (Promega) was added to the wells (100 μL) and cells were incubated at room temperature for 10 minutes. Lysates were transferred to an opaque white optical 96-well plate (10 μL) and the plate was read on a Berthold luminometer. The luminometer automatically injects a determined volume (50-100 μL) of Firefly substrate or Dual luciferase reagent (both from Promega) to each well and reads the luciferase activity for 1-10 sec. Raw data is exported in matrix format to an excel spreadsheet to be saved on the server. Alternatively, a one-step reagent (Promega Steady-Glo or Bright-Glo) was utilized. For this protocol cells were seeded directly onto a white opaque tissue culture plate (BD Bioscience) and stimulated as described above. Each well contained 100 μL of cells in media. An additional 25-100 μL of Promega reagent was added directly to each well and the plate was incubated for 5-30 minutes before luciferase quantification on the luminometer as described above.

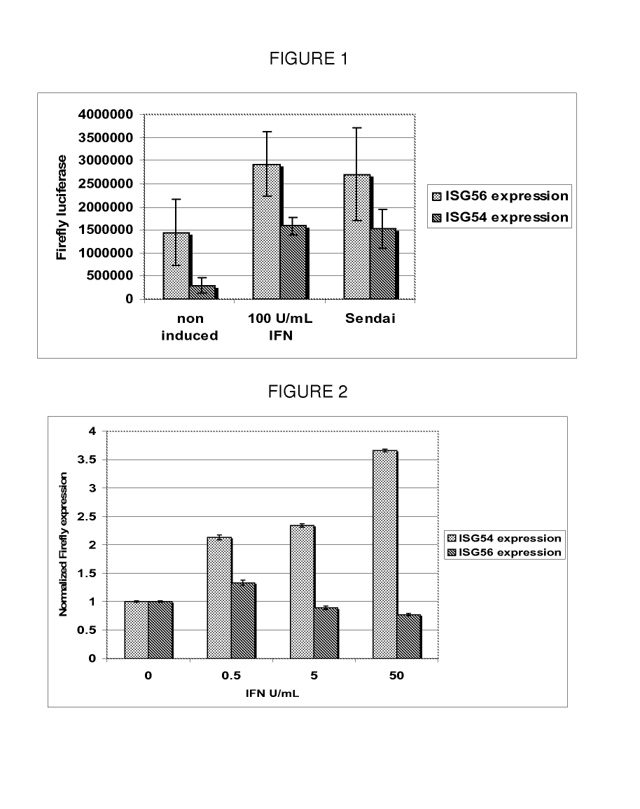

Reporter cell line synthesis. Huh7 cells were transiently transfected with reporter constructs containing the ISG54 or ISG56 promoters driving expression of firefly luciferase and tested for luciferase induction following Sendai virus infection or IFN treatment. Infection with Sendai virus causes activation of IRF-3 and binding/activation of ISRE sequences, whereas IFN only causes activation of ISRE sequences. ISG54 shows low basal levels of expression (no induction) and an increase in expression with Sendai or IFN treatment. Conversely, ISG56 shows higher levels of basal expression and only moderate induction with Sendai infection or IFN treatment (FIG. 1).

To address the high variability in the transient expression studies (most likely due to differences in transfection efficiency) the expression levels were normalized using the actin control constructs. Huh7 cells were co-transfected with the actin and ISG 54 constructs and then analyzed for firefly and Renilla luciferase production using the Dual-Glo luciferase reagent (Promega). When the normalized luciferase expression is calculated by using the Fluc:Rluc ratio, induction levels are reproducible (demonstrated through smaller error bars shown in FIG. 2). Production of stable cell lines eliminates variability due to differences in transfection efficiency. To analyze the inducibility of the luciferase reporter, cell lines were tested under different concentrations of IFN. Cells expressing the ISG54 promoter (not the ISG56 promoter) were shown to exhibit a dose response to increasing amounts of IFN and to be responsive to a low concentration of IFN (0.5 IU/mL) making it an attractive cell line for screening purposes (FIG. 2).

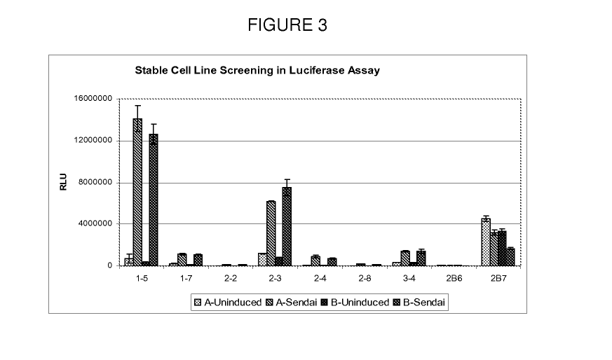

Huh7 cell lines that contain an integrated copy of the ISG54 reporter construct were clonally isolated and tested for luciferase expression when infected with Sendai virus as shown in FIG. 3. Luciferase induction was tested in two independent experiments done in triplicate wells to generate standard deviations. Cell line 2,4 was chosen for further characterization due to its low levels of basal expression and reproducible induction (15 fold over background). All ISG54 stable cell lines were passaged and frozen in a cell bank. Cell lines 2B6 and 2B7 are Huh7 cells that have an integrated copy of the actin promoter upstream of the Renilla luciferase gene (note these were the only two stable actin cell lines that were isolated). Both actin cell lines exhibit relatively low levels of uninduced expression and were further passaged for characterization.

Site-directed mutagenesis was performed on the ISG54 promoter to create optimal IRF3 binding sites and ISREs (interferon stimulated regulatory elements). Optimizing these sequences was predicted to result in better induction when stimulated with IFN or Sendai infection. Table 1 shows the sequence changes made in ISG54 and the resulting luciferase expression when transiently transfected into Huh7 cells. Results show that the endogenous ISG54 sequence provides the highest level of induction so the mutant constructs were not further characterized.

EXAMPLE 1 TABLE 1
Site-directed mutagenesis of ISG54-Luc to optimize activity.

| Clone | | Induction with |
|---|---|---|
| name | Sequence changes | Sendai infection |
| pKIN011 | ISG54 endogenous | 14 fold |
| pKIN016 | ISG54 A88G | 11 fold |
| pKIN017 | ISG54 3X ISRE | 7 fold |
| pKIN018 | A88G plus 3X ISRE | 7 fold |

Optimization of assay conditions: The growth and assay parameters were tested to identify the optimal conditions for conducting the high throughput screen in the ISG54 cell line (2,4). Table 2 shows the assay parameters tested and the optimal conditions that were determined through detection of luciferase expression. The levels of induction following Sendai infection were used to analyze each parameter and the condition giving the highest level of induction was chosen as optimal for screening purposes. Cell viability using an MTS assay was also tested in the presence of increasing DMSO and showed that Huh7 cells grown in media containing >2% DMSO exhibit increased toxicity as well as decreased induction.

EXAMPLE 1 TABLE 2
Huh7-ISG54-Luc high-throughput screen optimization studies

| | | Optimized assay |
|---|---|---|
| Assay parameter | Evaluated range | condition |
| DMSO tolerance | 0.5-5% | 0.5% |
| Serum concentration | 0-10% | 10% |
| Cell plating density | 5,000-20,000 cells/ | 10,000 |
| (96-well plates) | well | |
| Positive controls | 0.1-1,000 U/mL IFN | 100 U/mL IFN- and |
| | 0.5-200 HA/mL Sendai | 50 HA/mL Sendai |
| | virus | |
| Endpoint reagent | 25-200% | 50% |
| concentration | | |
| Timing of endpoint read | 8, 16, 24, & 48 | 24 h post treatment |
| Luciferase reading | Incubation and read time | 10 minutes, 1 sec |
| conditions | | |
| Reproducibility | n = 3 replicate | n = 3 replicate |
| | plates on each of 3 | plates on each of 3 |
| | days | days |

Discussion: A stable cell line expressing firefly luciferase using the endogenous ISG54 promoter was chosen for identifying RIG-I agonists in the HTS. This cell line exhibits low levels of endogenous expression (background in the cell based screen) and high levels of induction (14 fold) following Sendai virus infection. Two stable cell lines expressing Renilla luciferase under control of the actin promoter were selected for low levels of basal expression and no response to Sendai or IFN exposure. Both actin cell lines are being further characterized for their response to agents that globally increase transcription levels. To optimize the assay parameters for carrying out the HTS various conditions affecting cell growth and ISG54 induction were tested. The optimal concentration of cells, serum, DMSO, positive controls (Sendai and IFN) and luciferase substrate were determined. These conditions are utilized to screen a small molecule library for RIG-I agonists.

### Example 2

### Screen of RIG-I Targeted Library in ISG54 Cell Lines

Introduction: A targeted library was formed using a computer modeling program to predict compounds that interact with the RIG-I repressor domain. From the initial screen 7 compounds were identified as activating ISG54 expression significantly above background. Initial hits were validated in three assays to determine dose response, cytotoxicity using a MTS assay and promoter specificity which eliminated any compounds that nonspecifically activated expression of the actin promoter. Compound hits were analyzed for IRF-3 nuclear translocation to confirm they were activating the RIG-I pathway. Additionally, molecules were confirmed to induce endogenous ISG expression both at the RNA and protein level.

Validated hits were then analyzed for antiviral properties against RNA viruses in cell culture, including hepatitis C virus (HCV) and Influenza A virus. Screening of this small compound subset confirmed that the disclosed cell based screening platform is capable of identifying validated ISG54 agonists that function through IRF-3 and result in antiviral activity.

Compound library dilutions and daughter plates: The NCI small molecule library was received at 10 mM compound in 100% DMSO in 96-well sealed plates and stored at −20 degrees until used. Compound plates were thawed overnight at room temperature, aliquots of 2 mM diluted compounds (2 plates) were made in 96-well polystyrene plates, and plates were sealed with foil lids and stored at −20. Stock compounds were diluted 1:5 (10 mM to 2 mM) as follows: 5 μL of each stock compound was added to 20 μL of 100% DMSO. All plates were bar-coded and labeled with the original NCl identification system; the first row contains DMSO alone.

Screening compounds in ISG54-reporter cell line. Huh7-15G54-Luc cell lines are grown under selection conditions. Aliquots of cells were frozen under liquid nitrogen at 1×106 cells/vial or 3×106 cells per vial to be used in the experiments. Cell vials are removed from liquid nitrogen and grown in a T25 flask until 80% confluent (about 3 days) And are then expanded into a T75 flask until confluent (3 days). Cells are seeded in white opaque 96-well plates at a density of 1×10⁴ cells per well and grown for 24 hours without antibiotic selection. Each assay plate has wells A1-A4 treated with 0.5% DMSO containing media and wells A5-A8 infected with 10 hemagglutinin (HA) Sendai virus. The remainder of the plate is treated with 10 μM compound in media containing 0.5% DMSO.

To reach a final concentration of 10 μM the daughter plates of 2 mM compound are thawed at room temperature and the following dilution protocol is performed: From the daughter plate 10 μL of compound is transferred to a polystyrene 96-well plate containing 90 μL media and mixed thoroughly. From this dilution plate 10 μL of compound is transferred to a white opaque 96-well plate containing Huh-ISG54-Luc cells and 90 μL of media and mixed by pipetting. Cell plates are returned to incubator and grown for 24 hours. Steady-Glo luciferase reagent (Promega) is thawed, prepared as manufacturer directed and 50 μL of reagent is added to each well on cell plate directly (no media is removed). Cell plates are incubated at room temperature for 20 minutes and then read on the luminometer (Berthold) as described in Example 1.

Screening compounds in actin control cell line: Compounds that were identified as hits in the ISG54 reporter screen are plated on a hit plate at their original concentration of 2 mM. The actin cell line is grown and compounds are added in the manner as described above. The Dual-Glo luciferase reagent (Promega) was prepared as manufactured directed and 50-100 μL of reagent was added to each well on cell plate directly (no media is removed). Cell plates were incubated at room temperature for 10 minutes and then read on the luminometer (Berthold) as described in Example 1.

Dose dependency of ISG54-Luc reporter assay. The luciferase assays are performed as described above. To test for concentration dependency compounds dilution of 1, 5, 10, 20 and 50 μM are made in media containing a final concentration of 0.5% DMSO. The dilutions in media are made just prior to use and the compounds are not stored in this state. Only compounds in 100% DMSO are frozen and used in subsequent experiments.

MTS assay to determine cytotoxicity. Cultured human Huh7 cells are treated with increasing amounts of compound or equivalent amounts of DMSO diluted in media for 24 hours to see their effect on cell viability. The proportion of viable cells is calculated using a cell viability assay that measures conversion of a tetrazolium compound [3-(4,5-dimethyl-2-yl)-5-(3-carboxymethoxyphenyl)-2-(4-sulfophenyl)-2H-tetrazolium, inner salt; MTS] to a colored formazan compound in live cells. The conversion of MTS to formazan is detected in a 96-well microtiter plate reader, and the resulting optical densities can be plotted directly to estimate cell viability. Cell Titer One (Promega) is the one step reagent used as manufacturers protocol suggests and cells are incubated for three hours in the presence of reagent before O.D. reading is done. Compounds were diluted to final concentrations of 0, 5, 10, 20, and 50 μM in media containing 0.5% DMSO. Negative control wells contain no compound and positive control for cytotoxicity is examined using an EMCV infection which causes 100% cytopathic effect. Each compound concentration and control is done in triplicate wells to generate error bars.

EMCV antiviral assay. Cultured human Huh7 cells are seeded at 1.5×10⁴ cells/well and are pretreated with compound or equivalent amounts of media containing DMSO (negative control) for 24 hours. Then each well is infected with 250 pfu EMCV and incubated for 18 hours under normal growth conditions. Positive control wells are treated with 50 IU/mL Intron A. The level of viable cells is calculated using a cell viability assay that measures conversion of a tetrazolium compound [3-(4,5-dimethyl-2-yl)-5-(3-carboxymethoxyphenyl)-2-(4-sulfophenyl)-2H-tetrazolium, inner salt; MTS] to a colored formazan compound in live cells. The conversion of MTS to formazan is detected in a 96-well microtiter plate reader, and the resulting optical densities can be plotted directly to estimate cell viability. Cell Titer One is used as described above.

IRF-3 nuclear translocation assay. Huh7 cells are seeded in regular 96-well cell culture plates at a density of 5×10³ cells per well. Cells are grown under normal conditions for 24 hours. Compound plates (2 mM in 100% DMSO) were thawed at room temperature for 1-2 hours and then diluted into media. Compounds were diluted 1:10 and 1:20 in regular media and then 10 μL was added to the cell plate containing 90 μL of media in each well (this accounts for an additional 1:10 dilution). The final concentrations of these dilutions are 20 μM and 10 μM and the final amount of DMSO is 1% and 0.5% respectively. Negative control cells contain 0.5% DMSO in media and positive control cells are infected with 100 HA of Sendai virus for 24 hours.

Cells are incubated in the presence of compound for 20-24 hours and then monolayers are fixed, permeabilized and stained for IRF-3 protein. The staining protocol is performed according to the Cellomics translocation kit (Thermo Fisher) protocol and all buffers from the kit are used. IRF-3 specific serum obtained from commercial sources (Cell signaling #4962 and Zymed #39-2700) or polyclonal rabbit serum is used. The example described as follows is done using IRF-3 rabbit serum: Rabbit serum IRF-3 is diluted 1:400 in wash buffer and 100 μL is placed in each well to be stained. Secondary rabbit antibody conjugated with Dylight 488 and Hoescht dye (nuclear stain) are diluted in wash buffer and incubated as specified in the Cellomics protocol. Following secondary antibody incubation the monolayers are washed and left in 100 μL of wash buffer for imaging. IRF-3 FITC and nuclear staining (DAPI) are viewed on an inverted scope. Images are taken using MetaMorph software and saved as tif images in Powerpoint. All images are taken with the same exposure time. Additionally, high-throughput assay of IRF-3 nuclear translocation is performed with the ArrayScan instrument (Thermo Fisher). A 96-well plate is scanned and evaluation of IRF-3 nuclear translocation is performed using the same parameters for the entire plate.

HCV immunofluorescence antiviral assay: Huh7 cells are seeded on a 96-well plate at a density of 5×10³ cells per well and grown for 24 hours. Compounds that have been diluted to 10 μM in media and contain a final concentration of 0.5% DMSO are added to each well and grown another 24 hours. The compound media solution is removed from the plate and stored in a clean tissue culture dish. Cell monolayers are washed with PBS and HCV2a virus is added at the stated MOI. Virus is incubated for 2-4 hours and then removed, the monolayers are washed with PBS and the compound solutions are replaced into each well.

The cells are grown overnight and then cells are fixed and stained for HCV proteins. All buffers and reagents used are from the Cellomics staining kits described above. HCV specific antibodies from either commercial sources or primary patient serum can be used to detect HCV infected cells in culture. The example provided below uses primary patient serum: Serum is diluted 1:3,000 in wash buffer and incubated at room temperature for 1 hour. The secondary anti-human Dylight 488 or FITC Alexa 488 and Hoescht nuclear stain are diluted as stated above. Cells are washed and 100 μL of wash buffer is left in each well. Cellular staining is observed on an inverted microscope and images are taken as described above. The number of infected cells is counted and representative images are saved.

Influenza A virus ELISA assay: A549, MRC-5 or other cells permissive to Influenza virus infection are seeded in a 96 well plate at a density of 1×10⁴ cells/well. Cells are grown for 16 hours and compounds that were diluted to 5, 10, 20, 50 μM in media containing 0.5% DMSO are added to each well. Cells are incubated for 6 hours and then infected with 250 pfu Influenza WSN strain. Diluted virus is added directly to the well and compound is not removed. Infected cells are grown for a total of 24 hours post compound treatment and then fixed. The WSN Influenza ELISA protocol is done as follows: Cells are washed with PBS, fixed with methanol:acetone for 10 minutes and washed again with PBS. Cells are blocked with horse serum and BSA in the presence of Triton X-100. The primary antibody is mouse monoclonal anti-Influenza A nucleoprotein (Chemicon) and used at a 1:3000 dilution. The secondary antibody is goat anti-mouse IgG-HRP (Pierce) also used at a 1:3000 dilution. The reaction is developed using TMBK BioFX reagents as suggested. Following reagent addition the cells are incubated at room temperature for 2-5 minutes and 2N HCl is used to stop the reaction. Plates are read at 450 nM.

The results of the experiments described above are discussed below.

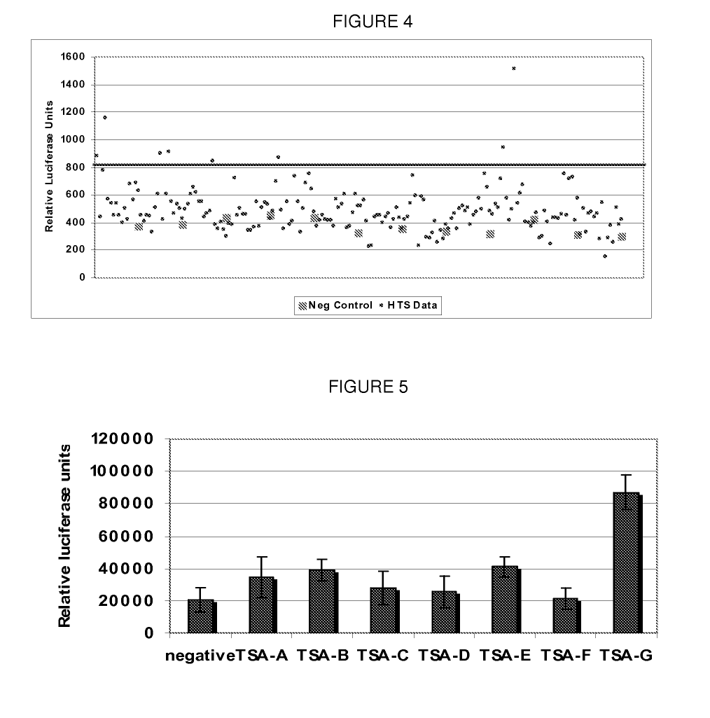

Identifying lead compounds in the Huh7-ISG54-Lucreporter cell line: Compounds in the RIG-I targeted set (168 molecules) were screened for activity in the Huh7-ISG54-Luc cells to identify RIG-I pathway agonists. FIG. 4 shows the scatter plot of all compounds screened and the line depicts the threshold set for identifying a molecule which significantly activates luciferase expression. From the targeted library subset 7 compounds activated ISG54 expression over 800 relative luciferase units and were chosen for further study (4.2% of library). Each plate contained negative controls (4 wells that are contain 0.5% DMSO in media but no compound) and positive controls (4 wells that were infected with Sendai virus and result in ISG54 induction). The controls on each plate are analyzed and the plate is repeated if necessary; however, no plates in the first library screen were repeated. FIG. 4 shows a scatter plot of initial hits from the targeted library. Negative controls (no treatment-gray) and positive controls (Sendai infection—not shown) were included on each plate. The luciferase values for all compounds screened are shown in red. The line represents the threshold for identifying initial hits.

To determine the specificity of induction the 7 initial hits were screened in a control cell that expresses luciferase using the actin promoter. The actin counter screen (FIG. 5) only showed one compound with increased activity of the actin promoter compared to background expression levels. The remainder of the compounds did not show any activation in the actin control cell line and all compounds were further validated. Lead compounds were chosen from original compound daughter plates (2 mM), transferred to a new polypropylene plate and serial dilutions were made. Final compound concentrations of 50 μM, 20 μM, 10 μM, and 5 μM were made in 0.5% DMSO containing media and were added to Huh7-ISG54-Luc cells to detect if the activation of ISG54 expression was dose dependent. Negative control wells contained Huh7-ISG54-Luc cells grown in media containing DMSO and positive control cells were infected with 100 HA Sendai virus.

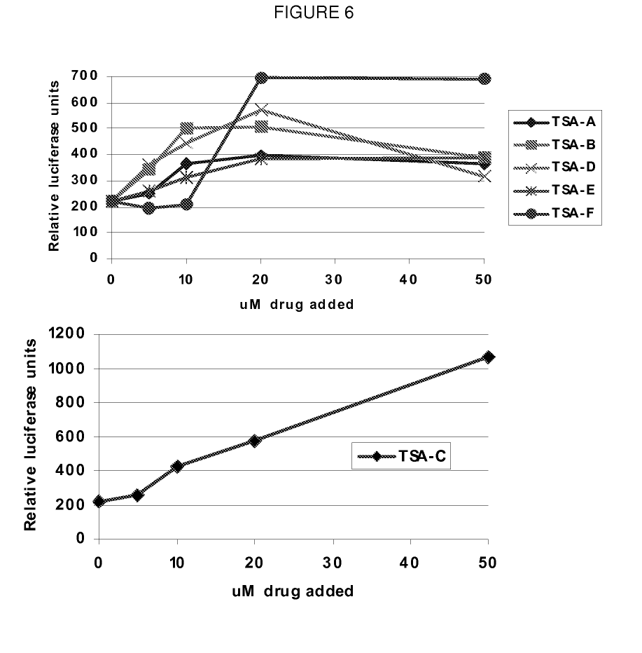

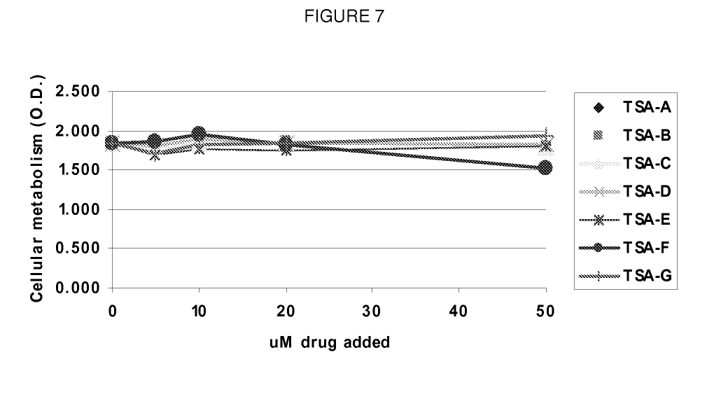

Additionally, the same compound dilutions were added to Huh7 cells to examine cytotoxicity in a MTS assay. Cells were treated for 24 hours with varying concentrations of compound and were analyzed for cell viability compared to negative control samples that were not treated with compound but grown in DMSO containing media. FIG. 6 shows dose dependent activity of compounds from the targeted set in the ISG54 reporter assay. FIG. 7 shows analysis of compound hits for initial cytotoxicity in the MTS assay. Interestingly all initial hits selected from the targeted set were confirmed to induce dose dependent activation of the ISG54 promoter and have no significant toxicity demonstrating a 100% validation rate of these targeted RIG-I compounds.

Mechanism of action and antiviral properties of lead compounds. Targeted set compounds that were validated as specifically activating the ISG54 promoter in a dose dependent manner without causing cytotoxicity in a MTS assay were examined for IRF-3 activation. It has been well described that upon activation of the RIG-I pathway IRF-3 becomes activated and translocates to the nucleus where it functions to up-regulate the transcription of several immune regulatory genes.

Huh7 cells were treated with compound or infected with Sendai virus as a positive control for 24 hours and subsequently stained for IRF-3. Rabbit serum was produced against recombinant IRF-3 protein and used to stain for IRF-3 in immuno-fluorescent assays.

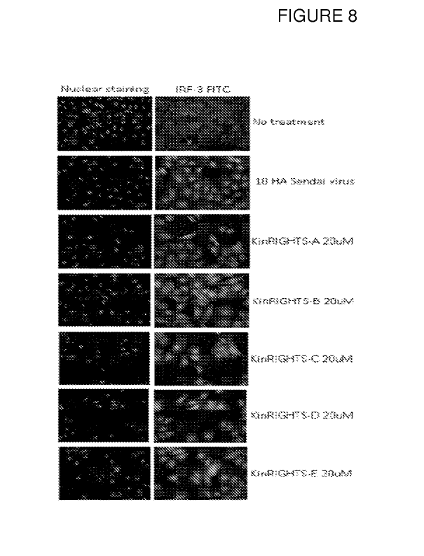

FIG. 8 shows all validated RIG-I targeted compounds displayed IRF-3 nuclear translocation in Huh7 cells. Negative control cells are treated with a similar concentration of DMSO and Sendai virus infected cells are a positive control for IRF-3 translocation. Additionally, compounds that did not activate ISG54 expression were used as negative controls and two negative compounds did not cause nonspecific changes in IRF-3 cellular localization. The intensity of IRF-3 within the nucleus varies for each compound and suggests that some have more activity than others.

Several of the compounds at lower concentrations (5 μM) did not cause IRF-3 activation and suggest that this measure of activity is dependent on compound concentration. By analyzing the activation of IRF-3 in liver cells it was confirmed that the ISG54 induction caused by these compounds is through the pathway that was intentionally targeted. In summary all ISG activating compounds from the RIG-I targeted set of compounds showed high levels of IRF-3 translocation as expected from molecules that bind to and activate the RIG-I receptor.

FIG. 8 shows IRF-3 translocation in Huh7 cells treated with compound. Cells were pre-treated with 10 μM of compound for 24 hours and then stained for IRF-3. Mock treated cells showed the majority of IRF-3 in the cytoplasm, Sendai infected cells have accumulated IRF-3 in the nucleus and compounds showed IRF-3 in the nucleus as well.

Antiviral characterization of validated compounds. Compounds were initially examined for antiviral activity against Influenza A virus-WSN strain (IFA) using an ELISA method to detect levels of infection. In both A549 cells and MRC5 cells which are both permissive to Influenza A virus infection none of the RIG-I agonist compounds from the targeted set had any significant activity. Cells were pre-treated with compound for either 8 hours or 24 hours and then infected with IFA virus. Cells were stained for viral protein to measure the level of infection.

Cells treated with compounds that induced IRF-3 translocation did not have any significant decrease in Influenza virus infection compared to cells that are mock treated. IRF-3 agonists that have been identified in other compound sets such as diversity libraries have shown good activity against Influenza virus using this assay. This data suggests that compounds targeted to bind and activate the RIG-I receptor directly may not have good efficacy against Influenza virus probably due to the high degree of innate immune response down-regulation following IFA infection.

Compounds were also examined for antiviral activity against Hepatitis C virus. To analyze antiviral activity cells were pre-treated with compounds at 10 μM (the concentration used to identify initial ISG54 activation) and subsequently infected with HCV2a purified virus. HCV2a was synthesized from a constructed clone amplified in Huh7 cells and concentrated to obtain high viral titers. The virus used in these experiments was approximately 5×10⁵ pfu/mL and the antiviral experiments used an MOI of 0.1-0.5.

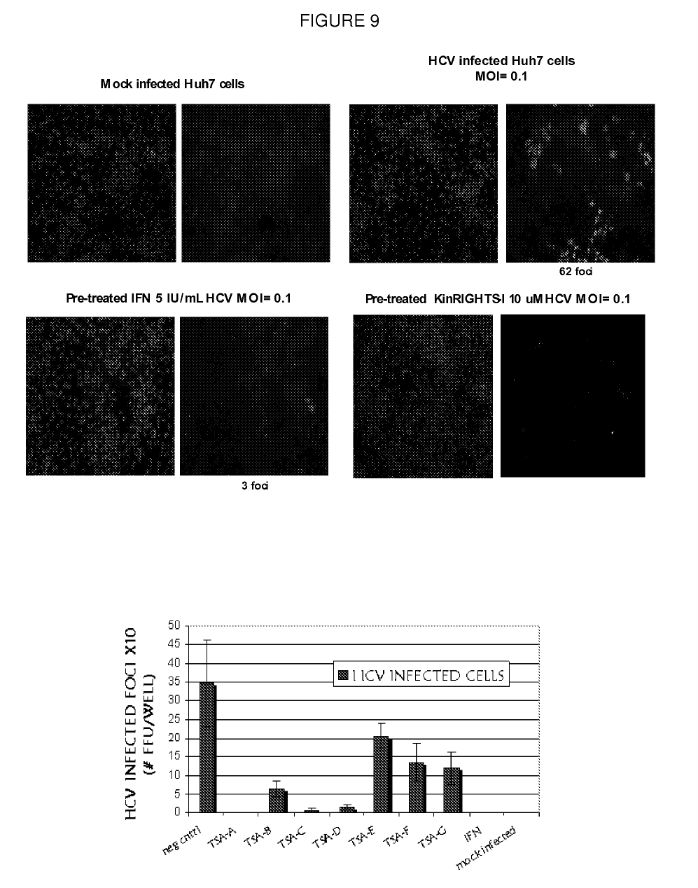

To measure levels of HCV infection with or without drug treatment the cells were stained for HCV specific staining using serum and a FITC conjugated fluorescent secondary antibody. HCV protein staining is specific, shows low background in mock infected cells and stains only the cytoplasm of cells where HCV replication occurs (FIG. 9, top panel). Using an inverted fluorescent microscope the number of infected cells is quantitated (shown—FIG. 9 bottom panel). Interferon treatment is used as a positive control and completely blocks HCV infection. A negative control compound that did not cause IRF-3 translocation was used to show that the antiviral activity is not due to treatment with any small molecule. This experiment provides evidence that RIG-I agonists identified which function through IRF-3 can inhibit HCV infection.

FIG. 9 shows HCV antiviral activity in the IF assay. Huh7 cells were pre-treated with compound for 24 hours, infected with HCV at a low MOI for 48 hours and then stained for HCV proteins. Mock infected cells showed no background staining, and interferon completely blocks infection and serves as a positive control. The number of infected cells (stained green for HCV proteins) are counted on an inverted microscope. The number of HCV infected cells after treatment for each compound is shown in the chart.

Several compounds from the targeted library showed high levels of HCV inhibition similar to IFN treated cells and were further examined in the HCV model. To confirm the specificity of antiviral activity HCV infection was examined following treatment with increasing concentrations of drug.

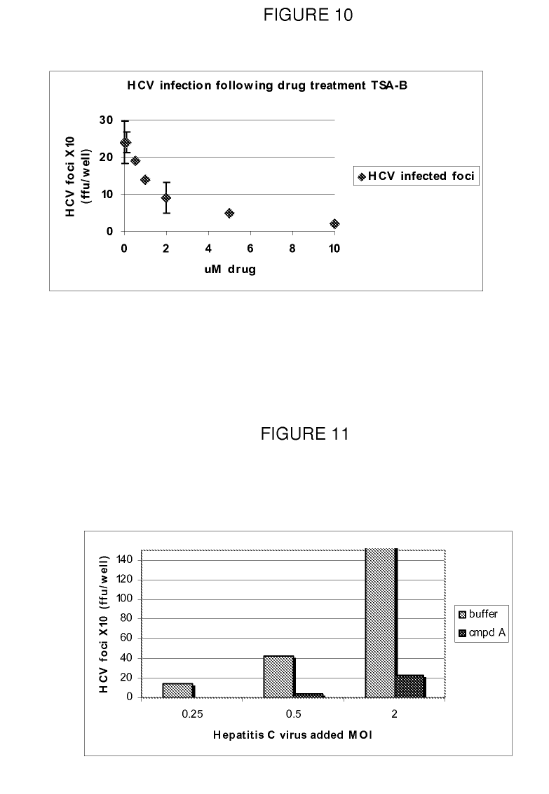

FIG. 10, Huh7 cells were pre-treated with compound at increasing concentrations 0-10 μM for 24 hours. Cells were then infected and analyzed for HCV foci as described above. FIG. 10 shows confirmation of one antiviral compound that has dose-dependent activity against HCV infection. Additionally, compounds were analyzed for antiviral activity against HCV with increasing MOI of virus added.

FIG. 11 shows one molecule that can inhibit HCV infection under conditions of high multiplicity of infection. Huh7 cells were treated with 10 μM of compound for 24 hours and subsequently HCV infections were done as described above.

In summary, screening of the RIG-I receptor targeted set of small molecules showed that molecules predicted to bind to the RIG-I receptor were identified as validated RIG-I agonists and a subset had antiviral properties. The initial hit rate of approximately 4% is higher than expected from a diversity set, as expected from a targeted molecule library. Additionally, the counter screening and validation assays proved a very small percentage of initial hits were false positive molecules and the validated hit rate was high. The validation assays utilized described in this Example were successful in identifying RIG-I agonists.

A series of antiviral systems were used to determine the antiviral properties of RIG-I agonist compounds using cell based systems to study HCV and Influenza A virus proving a subset of compounds to have dose dependent activity against HCV infection. As expected not all RIG-I agonists had antiviral properties due to viral countermeasures that can shut down pathway activation when in the presence of viral proteins. The RIG-I targeted library is utilized for additional screening due to the success in identifying validated agonist molecules and hits are further developed for their activity against HCV.

### Example 3

### Example 3A

### Identification OF RIG-I Agonists

To identify RIG-I agonists, a screening platform was used consisting of Huh7 cells harboring a luciferase reporter gene under the control of the ISG54 promoter. This promoter encodes tandem IRF-elements that bind activated IRF-3 (a RIG-I effector molecule) and an interferon (IFN)-stimulated response element that confers promoter induction by IFN-α/β. The assay conditions were optimized to yield low background under unstimulated conditions and reproducibly high levels of dose-dependent induction with positive control treatment such as Sendai virus infection. A small-molecule diversity library was selected to contain maximally diverse and drug-like compounds for agonist identification.

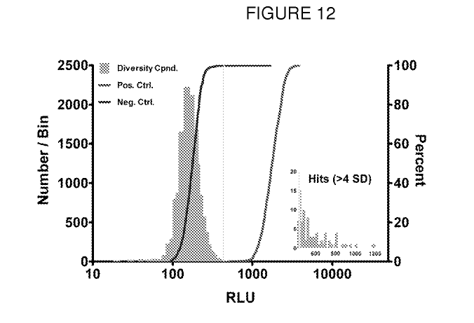

The results from the primary screen to identify molecules that induce ISG promoter activity are shown FIG. 12. A 20,000-member small molecule diversity library was screened at 10 μM to identify compounds that induce ISG54 luciferase reporter activity (grey histogram, 1° Y axis). Negative (cells alone) and positive controls (Sendai virus infected cells) are represented as cumulative frequency histograms (2° Y axis). Yellow line indicates the 4 SD threshold used to identify positive hits (inset). RLU refers to Renilla luciferase.

Only molecules that activated luciferase activity to four standard deviations above the mean (yellow line) over the entire diversity library were selected for further validation. Under these conditions, the initial hit rate was 0.49%, resulting in approximately 100 hits for further validation.

In addition to screening the diversity library, computational docking studies were used to identify small molecules that are predicted to bind to the ligand-binding domain of RIG-I. This “targeted set” was subsequently evaluated using the ISG54-luciferase screening cell line, yielding a 4% hit rate and demonstrating significant enrichment over the diversity screen. Several of these compounds were fully validated and demonstrate antiviral activity, described below.

### Example 3B

### Cytotoxicity and Antiviral Effects

All lead molecules chosen for further development mediate IRF-3 nuclear translocation and have antiviral activity against HCV. All induce dose-dependent activation of ISG expression in the absence of nonspecific promoter (β-actin) induction. To analyze in vitro cytotoxicity of lead agonist molecules, an MTS assay was conducted using multiple cell types (Huh7, human liver; MRC5, lung fibroblast; and 293, fibroblast cells). None of the compounds had a significant effect on cellular metabolism or metabolism as measured by the MTS assay at 50 μM.

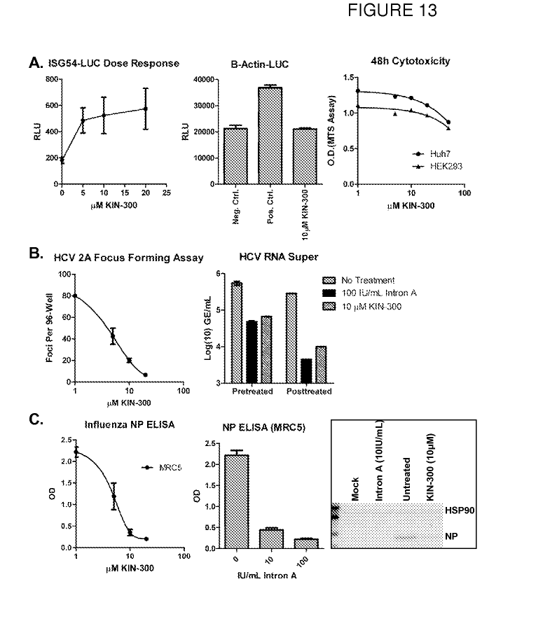

FIG. 13 shows characterization of exemplary compound KIN300, isolated from the diversity screen. As shown in FIG. 13A, initial hits were validated by demonstrating dose-dependent induction of the ISG54-luciferase reporter (left), absence of nonspecific promoter induction (β-actin-LUC, middle) and absence of cytotoxicity in multiple cell types (MTS assay, right).

FIG. 13B shows antiviral characterization, measured by inhibition of HCV focus formation (left) and viral RNA production in the supernatant (right) of Huh7 cells infected with a synthetic HCV 2A virus in combination with pre- or post-infection drug treatment. As shown in FIG. 13C, influenza studies characterized viral nucleoprotein production by ELISA (left) or Western blot (right) in drug-treated MRC5 cells infected with A/WSN/33 virus in comparison to control concentrations of IFN α-2a (Intron A, middle).

Compound inhibition on HCV infection was dose-dependent in a focus-forming assay, and this assay was used to calculate the 50% inhibitory concentration (IC50) for HCV infection (Table 1 and FIG. 13B). To examine compound inhibition on HCV replication and viral spread, viral RNA was measured in the supernatants of infected Huh7 cells by qPCR following drug pretreatment (FIG. 13B). Lead compounds caused a >1 log decrease in HCV RNA levels similar to treatment with 100 IU/mL pharmaceutical IFN-α (IntronA®, FIG. 13B). The compounds caused a similar decrease in HCV RNA levels when added post-infection, demonstrating antiviral activity in an established infection.

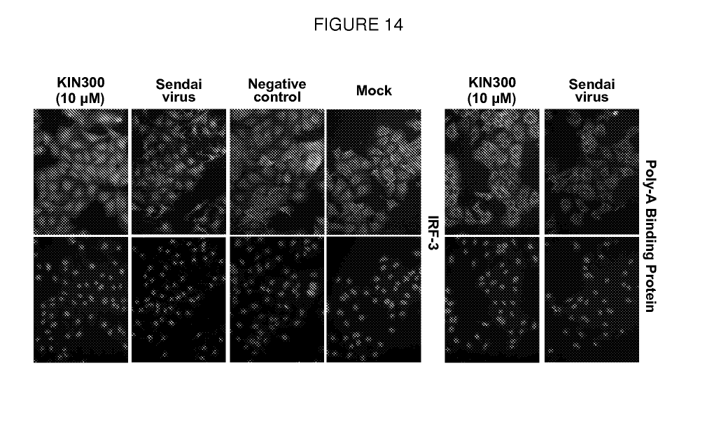

To examine compound effects on influenza virus infection, viral nucleoprotein (NP) levels were assayed by ELISA and Western blotting following drug treatment of infected cells, as shown in FIG. 13B. However, all molecules identified from the diversity library (KIN300, KIN400, and KIN500) exhibited efficient, dose-dependent anti-influenza activity (Table 1 and FIG. 14).

### Example 3C

### IRF-3 Nuclear Translocation

The induction of ISG expression mediated by RIG-I is conferred by phosphorylation, dimerization, and nuclear translocation of the IRF-3 transcription factor. Because Huh7 cells lack other pathogen-associated molecular pattern (PAMP) receptors to induce IRF-3, nuclear accumulation of the transcription factor is a specific indicator of RIG-I pathway activation in these cells (10). In normal un-stimulated Huh7 cells, IRF-3 shuttles between the cytoplasm and the nucleus resulting in diffuse cellular staining. Upon activation of the pathway by Sendai virus, IRF-3 translocates and accumulates in the nucleus.

IRF-3 (FIG. 14, left panels) was examined in Huh7 cells 24 hours after treatment with KIN300, Sendai virus (positive control), or a negative control compound (10 μM) that did not induce ISG expression. IRF-3 was detected with rabbit polyclonal serum and a DyLight 488 secondary antibody (green) and nuclei were detected by Hoescht staining (blue). Poly (A) binding protein (FIG. 14, right panels) was examined as a negative control using a monoclonal antibody and Dylight 488 (green).

The lead agonist molecules all stimulated dose-dependent IRF-3 translocation to an extent similar to Sendai virus (FIG. 14), but did not alter the distribution of a control factor (Poly A binding protein). Negative control compounds from the diversity screen did not alter IRF-3 localization, demonstrating a specific effect of the lead molecules. All lead compounds also up-regulated endogenous ISG mRNA expression and protein production in 293 cells, confirming pathway activation and compound activity in other cell types at the native promoter.

In summary, it has been shown that small, drug-like molecules can activate the RIG-I pathway and promote IRF-3 nuclear translocation leading to an antiviral effect. These studies also show validation of an in silico model of the RIG-I repressor domain and its application to identify interacting small-molecule compounds that possess antiviral activity.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

### REFERENCES

1. Tan, S. L., Ganji, G., Paeper, B., Proll, S., and Katze, M. G. (2007) Systems biology and the host response to viral infection, Nat Biotechnol 25, 1383-1389.
2. Lee, J., Wu, C. C., Lee, K. J., Chuang, T. H., Katakura, K., Liu, Y. T., Chan, M., Tawatao, R., Chung, M., Shen, C., Cottam, H. B., Lai, M. M., Raz, E., and Carson, D. A. (2006) Activation of anti-hepatitis C virus responses via Toll-like receptor 7, Proc Natl Acad Sci USA 103, 1828-1833.
3. Horsmans, Y., Berg, T., Desager, J. P., Mueller, T., Schott, E., Fletcher, S. P., Steffy, K. R., Bauman, L. A., Kerr, B. M., and Averett, D. R. (2005) Isatoribine, an agonist of TLR7, reduces plasma virus concentration in chronic hepatitis C infection, Hepatology 42, 724-731.
4. Johnson, C. L., and Gale, M., Jr. (2006) CARD games between virus and host get a new player, Trends Immunol 27, 1-4.
5. Li, K., Chen, Z., Kato, N., Gale, M., Jr., and Lemon, S. M. (2005) Distinct poly(1-C) and virus-activated signaling pathways leading to interferon-beta production in hepatocytes, J Biol Chem 280, 16739-16747.
6. Loo, Y. M., Fornek, J., Crochet, N., Bajwa, G., Perwitasari, O., Martinez-Sobrido, L., Akira, S., Gill, M. A., Garcia-Sastre, A., Katze, M. G., and Gale, M., Jr. (2008) Distinct RIG-I and MDA5 signaling by RNA viruses in innate immunity, J Virol 82, 335-345.
7. Loo, Y. M., Owen, D. M., Li, K., Erickson, A. K., Johnson, C. L., Fish, P. M., Carney, D. S., Wang, T., Ishida, H., Yoneyama, M., Fujita, T., Saito, T., Lee, W. M., Hagedorn, C. H., Lau, D. T., Weinman, S. A., Lemon, S. M., and Gale, M., Jr. (2006) Viral and therapeutic control of IFN-beta promoter stimulator 1 during hepatitis C virus infection, Proc Natl Acad Sci USA 103, 6001-6006.
8. Saito, T., Hirai, R., Loo, Y. M., Owen, D., Johnson, C. L., Sinha, S. C., Akira, S., Fujita, T., and Gale, M., Jr. (2007) Regulation of innate antiviral defenses through a shared repressor domain in RIG-I and LGP2, Proc Natl Acad Sci USA 104, 582-587.
9. Saito, T., Owen, D. M., Jiang, F., Marcotrigiano, J., and Gale, M., Jr. (2008) Innate immunity induced by composition-dependent RIG-I recognition of hepatitis C virus RNA, Nature 454, 523-527.
10. Sumpter, R., Jr., Loo, Y. M., Foy, E., Li, K., Yoneyama, M., Fujita, T., Lemon, S. M., and Gale, M., Jr. (2005) Regulating intracellular antiviral defense and permissiveness to hepatitis C virus RNA replication through a cellular RNA helicase, RIG-I, J Virol 79, 2689-2699.
11. Yoneyama, M., Kikuchi, M., Natsukawa, T., Shinobu, N., Imaizumi, T., Miyagishi, M., Taira, K., Akira, S., and Fujita, T. (2004) The RNA helicase RIG-I has an essential function in double-stranded RNA-induced innate antiviral responses, Nat Immunol 5, 730-737.
12. Kawai, T., Takahashi, K., Sato, S., Coban, C., Kumar, H., Kato, H., Ishii, K. J., Takeuchi, O., and Akira, S. (2005) IPS-1, an adaptor triggering RIG-1- and Mda5-mediated type I interferon induction, Nat Immunol 6, 981-988.
13. Meylan, E., Curran, J., Hofmann, K., Moradpour, D., Binder, M., Bartenschlager, R., and Tschopp, J. (2005) Cardif is an adaptor protein in the RIG-I antiviral pathway and is targeted by hepatitis C virus, Nature 437, 1167-1172.
14. Seth, R. B., Sun, L., Ea, C. K., and Chen, Z. J. (2005) Identification and characterization of MAVS, a mitochondrial antiviral signaling protein that activates NF-kappaB and IRF 3, Cell 122, 669-682.
15. Xu, L. G., Wang, Y. Y., Han, K. J., Li, L. Y., Zhai, Z., and Shu, H. B. (2005) VISA is an adapter protein required for virus-triggered IFN-beta signaling, Mol Cell 19, 727-740.
16. Venkataraman, T., Valdes, M., Elsby, R., Kakuta, S., Caceres, G., Saijo, S., Iwakura, Y., and Barber, G. N. (2007) Loss of DExD/H box RNA helicase LGP2 manifests disparate antiviral responses, J Immunol 178, 6444-6455.
17. Lipinski, C. A., Lombardo, F., Dominy, B. W., and Feeney, P. J. (2001) Experimental and computational approaches to estimate solubility and permeability in drug discovery and development settings, Adv Drug Deliv Rev 46, 3-26
18. Banerjee, S., Li, Y., Wang, Z., and Sarkar, F. H. (2008) Multi-targeted therapy of cancer by genistein, Cancer Lett 269, 226-242.
19. Odaka, M., Kohda, D., Lax, I., Schlessinger, J., and Inagaki, F. (1997) Ligand-binding enhances the affinity of dimerization of the extracellular domain of the epidermal growth factor receptor, J Biochem 122, 116-121.
20. Philo, J. S., Wen, J., Wypych, J., Schwartz, M. G., Mendiaz, E. A., and Langley, K. E. (1996) Human stem cell factor dimer forms a complex with two molecules of the extracellular domain of its receptor, Kit, J Biol Chem 271, 6895-6902.
21. Philo, J. S., Aoki, K. H., Arakawa, T., Narhi, L. O., and Wen, J. (1996) Dimerization of the extracellular domain of the erythropoietin (EPO) receptor by EPO: one high-affinity and one low-affinity interaction, Biochemistry 35, 1681-1691.
22. Kato, H., Takeuchi, O., Sato, S., Yoneyama, M., Yamamoto, M., Matsui, K., Uematsu, S., Jung, A., Kawai, T., Ishii, K. J., Yamaguchi, O., Otsu, K., Tsujimura, T., Koh, C. S., Reis e Sousa, C., Matsuura, Y., Fujita, T., and Akira, S. (2006) Differential roles of MDA5 and RIG-I helicases in the recognition of RNA viruses, Nature 441, 101-105.
23. Yoneyama, M., Kikuchi, M., Matsumoto, K., Imaizumi, T., Miyagishi, M., Taira, K., Foy, E., Loo, Y. M., Gale, M., Jr., Akira, S., Yonehara, S., Kato, A., and Fujita, T. (2005) Shared and unique functions of the DExD/H-box helicases RIG-I, MDA5, and LGP2 in antiviral innate immunity, J Immunol 175, 2851-2858.
24. Lescuyer, P., Strub, J. M., Luche, S., Diemer, H., Martinez, P., Van Dorsselaer, A., Lunardi, J., and Rabilloud, T. (2003) Progress in the definition of a reference human mitochondrial proteome, Proteomics 3, 157-167.
25. Taylor, S. W., Fahy, E., Zhang, B., Glenn, G. M., Warnock, D. E., Wiley, S., Murphy, A. N., Gaucher, S. P., Capaldi, R. A., Gibson, B. W., and Ghosh, S. S. (2003) Characterization of the human heart mitochondrial proteome, Nat Biotechnol 21, 281-286.
26. Lutfalla, G., Holland, S. J., Cinato, E., Monneron, D., Reboul, J., Rogers, N. C., Smith, J. M., Stark, G. R., Gardiner, K., Mogensen, K. E., and et al. (1995) Mutant U5A cells are complemented by an interferon-alpha beta receptor subunit generated by alternative processing of a new member of a cytokine receptor gene cluster, EMBO J. 14, 5100-5108.
27. Zou, J., Chang, M., Nie, P., and Secombes, C. J. (2009) Origin and evolution of the RIG-I like RNA helicase gene family, BMC Evol Biol 9, 85.
28. Renard, P., Ernest, I., Houbion, A., Art, M., Le Calvez, H., Raes, M., and

Remacle, J. (2001) Development of a sensitive multi-well colorimetric assay for active NFkappaB, Nucleic Acids Res 29, E21.
29. Suthar, M. S., Ma, D. Y., Thomas, S., Lund, J. M., Zhang, N., Daffis, S., Rudensky, A. Y., Bevan, M. J., Clark, E. A., Kaja, M. K., Diamond, M. S., and Gale, M., Jr. (2010) IPS-1 is essential for the control of West Nile virus infection and immunity, PLoS Pathog 6, e1000757.
30. Barnard, D. L. (2009) Animal models for the study of influenza pathogenesis and therapy, Antiviral Res 82, A110-122.
31. Daffis, S., Samuel, M. A., Suthar, M. S., Gale, M., Jr., and Diamond, M. S. (2008) Toll-like receptor 3 has a protective role against West Nile virus infection, J Virol 82, 10349-10358.
32. Blight, J. J. et al., (2002) J. Virology 76:13001-13014.

1. A method of identifying a compound that modulates innate immunity, comprising the steps of:
contacting at least one cell comprising a reporter gene under the control of a gene promoter responsive to innate immune activation with at least one putative innate immune response modulating compounds; and
measuring reporter gene activation.

2. The method of claim 1 further comprising
selecting a compound that activates reporter gene expression above a selected threshold for further characterization.

3. The method of claim 2 wherein said further characterization includes measuring nuclear translocation of transcription factors responsive to innate immune activation.

4. The method of claim 3 wherein said measuring of nuclear translocation is by immunochemical assay.

5. The method of claim 2, wherein the selected threshold is four standard deviations above a control level.

6. The method of claim 1, wherein, prior to contacting said compound is structurally selected for predicted binding to the ligand-binding domain of RIG-I.

7. The method of claim 1 wherein said cells are eukaryotic cells.

8. The method of claim 7 wherein said eukaryotic cells are Huh7 cells.

9. The method of claim 1 wherein said reporter gene is luciferase.

10. A method comprising providing eukaryotic cells comprising a reporter gene under the control of a gene promoter responsive to innate immune activation for identifying compounds that modulate innate immune responses.

11. The method of claim 10 wherein said cells are eukaryotic cells.

12. The method of claim 11 wherein said eukaryotic cells are Huh7 cells.

13. The method of claim 10 wherein said reporter gene is luciferase.

14. The method of claim 12 wherein said reporter gene is luciferase.

15. A method of preventing or treating a viral infection in a vertebrate by administering a compound identified by contacting at least one cell comprising a reporter gene under the control of a gene promoter responsive to innate immune activation with at least one putative innate immune response modulating compounds to said vertebrate; wherein said viral infection is treated, reduced or prevented.

16. The method of claim 15 wherein said compound activates reporter gene expression above a selected threshold for further characterization.

17. The method of claim 15 wherein said compound induces nuclear translocation of transcription factors responsive to innate immune activation.

18. The method of claim 16, wherein the selected threshold is four standard deviations above a control level.

19. The method of claim 15 wherein said viral infection is by a virus within one of the following families: Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae.

20. The method of claim 15 wherein said viral infection is influenza virus, Hepatitis C virus, West Nile virus, SARS-coronavirus, poliovirus, measles virus, Dengue virus, yellow fever virus, tick-borne encephalitis virus, Japanese encephalitis virus, St. Louis encephalitis virus, Murray Valley virus, Powassan virus, Rocio virus, Iouping-ill virus, Banzi virus, Ilheus virus, Kokobera virus, Kunjin virus, Alfuy virus, bovine diarrhea virus, Kyasanur forest disease virus or human immunodeficiency virus (HIV).
